(19) United States
(12) Patent Application Publication
(10) Pub. No.: US 2025/0352133 A1
(43) Pub. Date: Nov. 20, 2025

(54) AN INTRAORAL SCANNING DEVICE AND METHOD OF INTRAORAL SCANNING DEVICE COMMUNICATION

(71) Applicant: 3SHAPE A/S, Copenhagen K (DK)

(72) Inventor: Anders Robert JELLINGGAARD, Copenhagen K (DK)

(73) Assignee: 3SHAPE A/S, Copenhagen K (DK)

(21) Appl. No.: 18/874,184

(22) PCT Filed: Jun. 16, 2023
(86) PCT No.: PCT/EP2023/066208

§ 371 Date: Dec. 12, 2024

(30) Foreign Application Priority Data
Jun. 17, 2022 (EP) 22179519.8

(51) Int. Cl. A61B 5/00; A61C 9/00; G16H 30/20; G16H 40/67; H04W 12/069; H04W 12/50

(52) U.S. Cl. CPC A61B 5/4547; A61B 5/0033; A61B 5/0062; A61B 5/0088; A61C 9/0006; G16H 30/20; G16H 40/67; H04W 12/069; H04W 12/50; A61B 2560/0431; A61B 2562/0233; A61B 2576/02

(57) ABSTRACT

Embodiments relate to an intraoral scanning device that may include a processing unit configured to process intraoral scan data and provide 2D image data and/or 3D image data, a wireless interface configured for transmitting the 2D image data and/or the 3D image data, and a memory. The processing unit may be configured to receive a linking request for a session via the wireless interface, obtain a session identifier, transmit, via the wireless interface, a linking response. Furthermore, the processing unit may be configured to receive, via the wireless interface, an authentication message, select an intraoral scanning device key from a plurality of intraoral scanning device keys in the memory unit based on an authentication key identifier, verify client device data based on the selected intraoral scanning device key, and terminate the session if the verification fails.

### FIELD

The present disclosure pertains to the field of intraoral scanning devices, and in particular to intraoral scanning device security. Intraoral scanning device and method for secure intraoral scanning device communication is disclosed.

### BACKGROUND

The functionality of an intraoral scanning device becomes increasingly advanced. Wireless communication between an intraoral scanning device and external devices, such as a clinic computer, a scan computer, a dental software on a computer, and a customization computer, has evolved. Typically, a wireless communication interface of an intraoral scanning device uses open standard-based interface. However, this poses many challenges in terms of security. An intraoral scanning device may assume any incoming data as legitimate, and may allow memory to be written or changed by an unauthorized party. Any such attacks may result in a malfunction of the intraoral scanning device, or a battery exhaustion attack.

However, an intraoral scanning device is a small device with strict constraints in terms of computational power, memory space, etc. Therefore, a device communicating with an intraoral scanning device cannot use an off-the-shelf security algorithm and protocol, at the risk of e.g. depleting the intraoral scanning device battery or degrading functions of the intraoral scanning device rendering the intraoral scanning quasi-useless.

Present intraoral scanning devices are part of a service infrastructure which includes communication between intraoral scanning devices, scan software for a specific service, and the provider of the service. The service could for example include manufacture of an aligner, a retainer, a crown, an implant, a bracer, a nightguard etc. For improving the usability of such an infrastructure for the dentist, minimal interaction between the infrastructure and the dentist is needed. One way of achieving this is by applying wireless communication between the intraoral scanning device and an external computer that is connected to a server that can forward the intraoral scan data to a service provider. Scan data of a patient can be characterized as being personal information, and therefore, there is a need for minimizing any risk of a third party stealing or corrupting the at least scan data. The scan data is characterized as personal information, and in some situations, other type of personal information is associated with the scan data, such as age, gender, location address, personal security number etc. In this example, a demand for improving the security of the wireless communication in the service infrastructure is needed.

### SUMMARY

An aspect of the present disclosure is to reduce risk of a third party accessing any part of the intraoral scanning device. There is a need for an intraoral scanning device that is protected against unauthorized modification of the intraoral scanning device and operation thereof.

A further aspect of the present disclosure is to provide an intraoral scanning device, and a method which seeks to mitigate, alleviate, or eliminate a third party's possibility to steal and/or corrupt personal information of the patient.

An even further aspect of the present disclosure is to improve security of an intraoral scanning device. Namely, the intraoral scanning device disclosed herein is robust against security threats, vulnerabilities and attacks by implementing appropriate safeguards and countermeasures, such as security mechanisms, to protect against threats and attacks. The present disclosure relates to an intraoral scanning device that is robust against replay attacks, unauthorized access, battery exhaustion attacks, eavesdropping and man-in-the-middle attacks.

An even further aspect of the present disclosure is to provide the intraoral scanning device the capability of securing access thereto from unauthenticated parties, and securing its communication against modification attacks and replay attacks while minimizing computational overhead and power consumption of the intraoral scanning device. Furthermore, the present disclosure provides a scalable security architecture.

According to the aspect, an intraoral scanning device configured to acquire intraoral scan data from a three-dimensional dental object during a scanning session is disclosed. The intraoral scanning device may comprise a processing unit configured to process intraoral scan data of a patient and provide 2D image data and/or 3D image data, a wireless interface configured to transmit the 2D image data and/or the 3D image data, and a memory. The processing unit may be configured to receive a linking request for a session via the wireless interface, obtain a session identifier, transmit, via the wireless interface, a linking response comprising an intraoral scanning device identifier and the session identifier. Furthermore, the processing unit may be configured to receive, via the wireless interface, an authentication message comprising an authentication key identifier and client device data, select an intraoral scanning device key from a plurality of intraoral scanning device keys in the memory unit based on the authentication key identifier, verify the client device data based on the selected intraoral scanning device key, and terminate the session if the verification fails.

According to the aspect, a method for configuration of an intraoral scanning device that may comprise a processing unit configured to process intraoral scan data of a patient and provide 2D image data and/or 3D image data, a memory unit and a wireless interface configured for transmitting the 2D image and/or the 3D image. The method may comprise receiving a linking request for a session via the wireless interface, obtaining a session identifier, transmitting, via the wireless interface, a linking response comprising an intraoral scanning device identifier and the session identifier, receiving, via the wireless interface, an authentication message comprising an authentication key identifier and client device data, selecting an intraoral scanning device key from a plurality of intraoral scanning device keys based on the authentication key identifier, verifying the client device data based on the selected intraoral scanning device key; and terminating the session if verification fails.

According to the aspect, an intraoral scanning device for acquiring intraoral scan data from a three-dimensional dental object during a scanning session is disclosed. The intraoral scanning device may comprise a processing unit configured to process intraoral scan data of a patient and provide 2D image data and/or 3D image data, a wireless interface configured for transmitting the 2D image data and/or the 3D image data, and a memory. The processing unit may be configured to receive a connection request for a session via the wireless interface, obtain a session identifier, transmit, via the wireless interface, a connection response comprising an intraoral scanning device identifier and the session identifier. Furthermore, the processing unit may be configured to receive, via the wireless interface, an authentication message comprising an authentication key identifier and client device data, select an intraoral scanning device key from a plurality of intraoral scanning device keys in the memory unit based on the authentication key identifier, verify the client device data based on the selected intraoral scanning device key, and terminate the session if the verification fails.

The intraoral scanning device is a handheld scanning device for scanning inside an oral cavity of a patient. The intraoral scanning device differs from other type of teeth scanning device in that the intraoral scanning device is a handheld scanning device which can easily be handled by one hand by a user, and which has now wired connection to any external device during scanning of an inside of an oral cavity of a patient. Therefore, the only attack which an intraoral scanning device may experience is via the wireless interface.

The method and the intraoral scanning device as disclosed provide secure configuration of the intraoral scanning device, such as secure access to the memory of the intraoral scanning device. It is an advantage of the present disclosure that the intraoral scanning device can only be configured or updated by authorized parties. The disclosed intraoral thus has the advantage of detecting and preventing any modification by unauthorized parties. The intraoral scanning device disclosed herein is advantageously protected against attacks such as spoofing attacks, man-in-the-middle attacks, and/or replay-attacks.

The intraoral scanning device is the key element in providing the needed level of security in wireless communication in a service infrastructure which at least includes the intraoral scanning device and a scan computer or a dental software on a computer. It would not be possible for a third party to attack the wireless communication as this person needs to have the intraoral scanning device physically in its hand. It would not even be enough to have access to the scan computer or the dental software.

The method as disclosed herein provides a secure configuration and/or update of an intraoral scanning device.

The present disclosure provides improved security of an intraoral scanning device. Security comprises assessing threats, vulnerabilities and attacks and developing appropriate safeguards and countermeasures to protect against threats and attacks.

The intraoral scanning device comprises a processing unit. The processing unit may be configured to process intraoral scan data of a patient and provide 2D image data and/or 3D image data. The 2D image data and/or 3D image data may include information about the anatomy of the oral cavity of the patient, such as teeth, gingival, bone level, and/or information about diagnostic indicators such as caries, bone loss, gingivitis, gingiva recession, periodontitis, bone loss, cracks, and occlusion.

The 2D image data and/or the 3D image data may be image data configured to be visualizable on a display in a 2D or a 3D manner, respectively.

As used herein, the term “certificate” refers to a data structure that enables verification of its origin and content, such as verifying the legitimacy and/or authenticity of its origin and content. The certificate may be configured to provide a content that is associated to a holder of the certificate by an issuer of the certificate. The certificate comprises a digital signature, so that a recipient of the certificate is able to verify or authenticate the certificate content and origin. The certificate may comprise one or more identifiers and/or keying material, such as one or more cryptographic keys (e.g. an intraoral scanning device key) enabling secure communication in an intraoral scanning device system. The certificate permits thus to achieve authentication of origin and content, non-repudiation, and/or integrity protection. The certificate may further comprise a validity period, one or more algorithm parameters, and/or an issuer. A certificate may comprise a digital certificate, a public key certificate, an attribute certificate, and/or an authorization certificate.

As used herein, the term “key” refers to a cryptographic key, i.e. a piece of data, (e.g. a string, a parameter) that determines a functional output of a cryptographic algorithm. For example, during encryption, the key allows a transformation of a plaintext into a cipher-text and vice versa during decryption. The key may also be used to verify a digital signature and/or a message authentication code, MAC. A key is so called a symmetric key when the same key is used for both encryption and decryption. In asymmetric cryptography or public key cryptography, a keying material is a key pair, so called a private-public key pair comprising a public key and a private key. In an asymmetric or public key cryptosystem (such as Rivest Shamir Adelman, RSA, cryptosystem, and elliptic curve cryptography, ECC), the public key is used for encryption and/or signature verification while the private key is used for decryption and/or signature generation. The intraoral scanning device key may be keying material allowing deriving one or more symmetric keys, such as a session key and/or a certificate key for intraoral scanning device communication. The intraoral scanning device key may be stored in a memory unit of the intraoral scanning device, e.g. during manufacture. The intraoral scanning device key may comprise keying material that is used to derive a symmetric key. The intraoral scanning device key comprises for example an Advanced Encryption Standard, AES, key, such as an AES-128 bits key.

As used herein the term “identifier” refers to a piece of data that is used for identifying, such as for categorizing, and/or uniquely identifying. The identifier may be in a form of a word, a number, a letter, a symbol, a list, an array or any combination thereof. For example, the identifier as a number may be in the form of an integer, such as unsigned integer, uint, with a length of e.g. 8 bits, 16 bits, 32 bits, etc., such as an array of unsigned integers.

The term “client device” as used herein refers to a device that is able to communicate with the intraoral scanning device. The client device may refer to a computing device acting as a client. The client device may comprise a customization device, a relay, a tablet, a personal computer, an application running on a personal computer or tablet, and/or USB dongle plugged into a personal computer.

The present disclosure relates to an intraoral scanning device. The intraoral scanning device may comprise a processing unit configured to process intraoral scan data of a patient and provide 2D image data and/or 3D image data, a memory unit and a wireless interface. The memory unit may include removable and non-removable data storage units including, but not limited to, Read Only Memory (ROM), Random Access Memory (RAM), etc. The memory unit may have an intraoral scanning device certificate stored thereon. The memory unit may have the intraoral scanning device certificate stored at a memory address of the memory unit, and/or in memory cells of the memory unit, such as in designated memory cells and/or at designated addresses. The wireless interface may comprise a wireless transceiver, e.g. configured for wireless communication at frequencies in the range from 2.4 to 2.5 GHz, 2.4 GHz to 5 GHz, about 2.45 GHz or about 5 GHz. In one or more exemplary intraoral scanning devices, the wireless interface is configured for communication, such as wireless communication, with a client device or an intraoral scanning device, respectively comprising a wireless transceiver configured to receive and/or transmit data. The processing unit may be configured to receive a linking request for a session via the wireless interface; and to obtain a session identifier, e.g. in response to the linking request. The wireless interface may be configured to receive the linking request for a session from a client device. The processing unit may be configured to obtain a session identifier, such as by generating a random or pseudo-random number. The processing unit may be configured to store the session identifier in the memory unit. The memory unit may be configured to store the session identifier at a memory address of the memory unit, and/or in memory cells of the memory unit, such as in designated memory cells and/or at designated addresses. The linking request may comprise an authentication key identifier and/or an authentication type identifier, in order to permit the intraoral scanning device to perform authentication of the linking request and the client device sending the linking request at this early stage. This may provide a level of access control.

The processing unit may be configured to transmit via the wireless interface a linking response comprising an intraoral scanning device identifier and the session identifier. The processing unit may be configured to generate a linking response by including the session identifier and the intraoral scanning device identifier in the linking response. The intraoral scanning device identifier may refer to a unique identifier of the intraoral scanning device. The intraoral scanning device identifier may be included in the intraoral scanning device certificate. The wireless interface may be configured to transmit the linking response to e.g. the client device.

The processing unit may be configured to receive, via the wireless interface, an authentication message comprising an authentication key identifier and client device data. For example, the wireless interface may be configured to receive the authentication message from the client device. For example, the intraoral scanning device receives the authentication message from the client device in order to establish a communication session. The client device data may comprise a client device certificate (encrypted or unencrypted), customization data, intraoral scanning device operating parameters, and/or firmware data. For example, the authentication message may comprise an authentication key identifier in plain text. The authentication key identifier is indicative of an intraoral scanning device key, an intraoral scanning device key stored in the memory unit of the intraoral scanning device, for example as part of the intraoral scanning device certificate.

The intraoral scanning device operating parameters may corresponds to settings of the handheld intraoral scanning device that involves settings of the image sensor, light projector, the wireless interface, a scan sequence of the handheld intraoral scanning device. Etc. The scan sequence corresponds to a scanning of a patient's jaws with the handheld intraoral scanning device, while in real-time the handheld intraoral scanning device is configured to determine and transmit the 3D image data based on the intraoral scan data acquired by the image sensor of the handheld intraoral scanning device during the scan sequence.

Furthermore, the intraoral scanning device operating parameters relates to power management settings, configuration of a user interface of the intraoral scanning device and/or settings of an optical unit of the intraoral scanning device.

The handheld intraoral scanning device may include a user interface which may include at least a touch sensor, at least a touch button, at least a light emitting diode, a haptic sensor, and/or an accelerometer. The handheld intraoral scanning device may include a motion sensor which is configured to sense the motion of the handheld intraoral scanning device. The handheld intraoral scanning device is configured to communicate wirelessly with an external device that is connected to a display. A cursor on the display may be moved around based on motion signals provided by the motion sensor to the external device. The user is able to navigate the cursor on the display by moving the handheld intraoral scanning device. The session data may include settings update that relates to the motion sensor of the handheld intraoral scanning device, and the customization data may include settings for customizing a user interface of the handheld intraoral scanning device which may involve a graphical setup of a graphical user interface on the display. For example, when the handheld intraoral scanning device connects to the external device, the handheld intraoral scanning device forwards a customization package to the external device via the wireless interface, and the external device is then configured to change the graphical setup based on the customization package. The customization package may be updated by the customization data provided by the session data.

The firmware data may include updates to the handheld intraoral scanning device that improves the functionality and features of the device.

The processing unit may be configured to select an intraoral scanning device key from a plurality of intraoral scanning device keys in the memory unit, based on the authentication key identifier and optionally other identifiers. When the authentication key identifier is acceptable by the intraoral scanning device based on an intraoral scanning device key identifier held by the intraoral scanning device, the processing unit may be configured to select an intraoral scanning device key that the authentication key identifier indicates and to use the selected intraoral scanning device key as keying material in securing the session. The processing unit may be configured to select an intraoral scanning device key from a plurality of intraoral scanning device keys in the memory unit based on the authentication key identifier and an authentication type identifier.

The authentication type identifier may be received in plaintext by the intraoral scanning device, and/or as client device type identifier in the client device certificate (encrypted or decrypted). For example, the processing unit may be configured to select an intraoral scanning device key which the authentication key identifier and the authentication type identifier indicate.

The processing unit may be configured to verify the client device data, based on the selected intraoral scanning device key; and to terminate the session if verification fails. To verify the client device data may be based on an intraoral scanning device certificate or at least parts thereof. To verify the client device data based on the selected intraoral scanning device key may comprise verifying the integrity of the client device data based on the selected intraoral scanning device key, such as verifying a MAC and/or a digital signature comprised in the client device data. To verify the client device data based on the selected intraoral scanning device key may comprise decrypting the client device data, e.g. a client device certificate, using the selected intraoral scanning device key (as keying material to derive a decryption key or as a decryption key), when the client device data is received encrypted. To verify the client device data based on the selected intraoral scanning device key may comprise verifying the client device data, e.g. decrypted client device certificate, by comparing the received client device data with data stored in the memory unit. The client device data may comprise a client device certificate (such as an encrypted client device certificate), an authentication key identifier, and/or an authentication type identifier. The client device may be assigned a client device certificate. The client device certificate refers to a certificate generated and assigned to the client device by e.g. a device manufacturing the client device.

The client device certificate may comprise a certificate type identifier. The certificate type identifier may indicate a type of the certificate amongst a variety of certificate types, such as an intraoral scanning device family certificate type, an intraoral scanning device certificate type, a firmware certificate type, a research and development certificate type, client device certificate type. The certificate type identifier may be used by the intraoral scanning device to identify what type of certificate it receives, stores, and/or retrieves. The client device certificate may comprise a version identifier indicative of a data format version of the certificate. The intraoral scanning device may be configured to use the certificate type identifier and/or the version identifier to determine what type of data the certificate comprises, what type of data is comprised in a field of the certificate. For example, the intraoral scanning device determines based on the certificate type identifier and/or version identifier what field of the certificate comprises a digital signature and/or which public key is needed to verify the digital signature. It may be envisaged that there is a one-to-one mapping between the certificate type identifier and the public-private key pair.

The client device certificate may comprise a signing device identifier. The signing device identifier refers to a unique identifier identifying the device (such as a manufacturing device, e.g. an integrated circuit card, a smart card, a hardware security module) that has signed the client device certificate. The signing device identifier may for example comprise a medium access control, MAC, address of the signing device and/or a serial number. The signing device identifier optionally allows for example the intraoral scanning device to determine whether the signing device is e.g. black-listed or not, and thus to reject certificates signed by a signing device that is black-listed.

The client device certificate may comprise one or more hardware identifiers such as a first hardware identifier and/or a second hardware identifier. A hardware identifier may identify a piece of hardware comprised in the client device, such as a radio chip comprised in the client device or a digital signal processor of the client device. The hardware identifier may be stored in a register of the piece of hardware comprised in the intraoral scanning device during manufacturing of the piece of hardware. The hardware identifier may comprise a serial number, a medium access control, MAC, address, a chip identifier, or any combination thereof. The client device certificate may comprise a client device type identifier. A client device type identifier may be indicative of a type which the client device belongs to. The client device may be attributed a client device type corresponding to a model, category or type of client devices, such as a customization type, e.g. a computer product model, category or type configured for customizing the intraoral scanning device, a USB dongle product model, category or type configured for customizing the intraoral scanning device.

The client device certificate may comprise a client device identifier. The client device identifier refers to an identifier identifying a client device. The client device identifier may for example comprise a medium access control, MAC, address of the client device, and/or a serial number of the client device.

The client device certificate may comprise a client device key identifier. A client device key identifier may be indicative of the client device key used as keying material for securing a communication with an external party, such as with an intraoral scanning device. In one or more exemplary client device certificates, the client device certificate comprises a Bluetooth address or an IP address of the client device.

The client device certificate comprises a digital signature. The digital signature enables a proof or verification of authenticity of the intraoral scanning device certificate, such as verification of the signer legitimacy. The digital signature is optionally generated by the manufacturing device using a client device customization private key. The intraoral scanning device may be configured to verify the digital signature of the client device certificate when receiving the (encrypted or unencrypted) client device certificate comprising the digital signature (i.e. receiving the authentication message comprising the encrypted client device certificate, and obtaining a decrypted version of the client device certificate). The digital signature is verifiable by the intraoral scanning device using a corresponding client device customization public key. If the digital signature is not successfully verified using the alleged public key, the intraoral scanning device may disregard the client device certificate and/or abort normal operation. This may provide the advantage that the intraoral scanning device rejects a client device certificate that is tampered or received from unauthenticated parties. The communication with the intraoral scanning device may thus be robust against impersonation, modification and masquerading attacks.

The authentication message may comprise an authentication type identifier. To select an intraoral scanning device key from a plurality of intraoral scanning device keys may be based on the authentication type identifier. An authentication type identifier may be indicative of a client device type identifier and/or a certificate type identifier, e.g. of the (encrypted) client device certificate. The client device may be attributed a client device type corresponding to a model, category or type of client devices, such as a customization type, e.g. a computer product model, category or type configured for customizing the intraoral scanning device, a USB dongle product model, category or type configured for customizing the intraoral scanning device. A client device type identifier may refer to an identifier indicative of a client device type. A client device type identifier may uniquely identify a client device type. A client device type identifier may identify a type which the client device belongs to. The client device type identifier may be comprised in the client device certificate. The intraoral scanning device may be configured to select the intraoral scanning device key corresponding to the authentication type identifier and/or the authentication key identifier.

Customizing the intraoral scanning device implies that a customization part of the memory can be in read and/or writ mode. Customizing the intraoral scanning device implies that a firmware part of the memory is write-protected. The customization part of the memory may comprise setting data, such as power management settings, configuration of a user interface of the intraoral scanning device and/or settings of an optical unit of the intraoral scanning device.

The optical unit may include one or more light projectors, one or more optical components, and one or more image sensors.

The user interface of the intraoral scanning device may include at least a touch sensor, at least a touch button, at least a light emitting diode, a haptic sensor, and/or an accelerometer.

The client device data may include customization data which include setting data, such as power management settings, configuration of a user interface of the intraoral scanning device and/or settings of an optical unit of the intraoral scanning device. The client device data may include improved feature updates, new feature updates relating to an operating software system, a FPGA or other electronic/digital hardware of the intraoral scanning device.

The client device data may comprise an encrypted client device certificate; and the processing unit may be configured to generate a certificate key based on the selected intraoral scanning device key and/or the session identifier. To verify the client device data may comprise to decrypt the encrypted client device certificate with the certificate key to obtain a decrypted version of the encrypted client device certificate. The encrypted client device certificate may be generated by the client device using an encryption algorithm and a certificate key.

The intraoral scanning device may be configured to decrypt the encrypted client device certificate using a certificate key, a common secret and/or an intraoral scanning device key. The certificate key may be based on a common secret and/or a certificate value. The intraoral scanning device may be configured to obtain and/or generate the common secret based on an intraoral scanning device key, such as the selected intraoral scanning device key. For example, to generate the common secret based on the intraoral scanning device key, the intraoral scanning device may retrieve from the memory unit the intraoral scanning device key and/or the intraoral scanning device certificate from the memory unit, the intraoral scanning device certificate comprising an intraoral scanning device key, which is to be used for derive the common secret. The intraoral scanning device may be configured to store the common secret in the memory unit, so as to e.g. retrieve the common secret from the memory unit when needed.

The intraoral scanning device being configured to receive client device data or a linking request may be scheduled for a specific time on a day when the intraoral scanning device will not be used. The scheduling may be determined by the processing unit based on historical usage time of the intraoral scanning device and a machine learning model. The machine learning model receives timestamps from a clock in the intraoral scanning device and input information about when the intraoral scanning device is being used in a scanning session. The machine learning model includes a training data set which includes historical usage time of the intraoral scanning device being in the scanning session. Based on the machine learning model and a timestamp defining the time of the day the processing unit will know when to be configured to receive the client device data. The advantage of the scheduling is that a valid authenticated mode request will not interfere the work of the dentist with the intraoral scanning device. Furthermore, when being placed into the customization mode, the intraoral scanning device can be programmed to do time consuming updates within specific time-period(s). For example, an update which last more than 30 mins will automatically be planned to be performed in a time-period of more than 30 mins where the intraoral scanning device will not be used, such as outside the working hours or during a break of the dentist/clinic.

The processing unit may be configured to place the intraoral scanning device into the requested mode if authentication of the mode request succeeds and if a timestamp is within a time-period. The timestamp is generated by a clock of the intraoral scanning device and received by the processing unit.

The processing unit may include a machine learning model that includes a training data set which includes historical data that relates to usage time of the intraoral scanning device being in a scanning session, and wherein the machine learning model receives a timestamp from a clock in the intraoral scanning device and input information about when the intraoral scanning device is being used in a scanning session, and the processing unit may then be configured to receive a linking request or client device data based on an output of the machine learning model. The output of the machine learning model is a trigger for the processing unit to know when to be in a state for receiving a linking request and client device data The intraoral scanning device may be configured to generate the common secret based on a session identifier using the processing unit and to store the common secret in the memory unit. For example, the intraoral scanning device may generate a common secret based on an intraoral scanning device key, e.g. the selected intraoral scanning device key, and a session identifier. The intraoral scanning device may generate the common secret CS, e.g. as follows:

$$CS = hash ( HD\_KEY , S\_ID ) ,$$

where hash is a hash function, HD_KEY is the (selected) intraoral scanning device key and S_ID is a session identifier. The session identifier may be generated by the intraoral scanning device upon reception of a linking request. The session identifier may comprise a random or pseudo random number of a defined length. The common secret may be used as a certificate key in one or more exemplary intraoral scanning devices.

The certificate key may be based on the common secret, e.g. generated by performing a hash function on the common secret and/or a certificate value. The intraoral scanning device may then generate the certificate key e.g. as follows:

$$C\_KEY = hash ( CS , C\_VAL ) ,$$

where hash is a hash function, CS is the common secret and C_VAL is a certificate value. The certificate value may be a predefined value or string, such as “certificate”.

In one or more exemplary intraoral scanning devices, the certificate key may optionally be generated by performing a hash function on the intraoral scanning device key and the session identifier. The intraoral scanning device may decrypt the encrypted client device certificate (part of the client device data) using the certificate key generated by the intraoral scanning device and obtain the decrypted version of the client device certificate. The intraoral scanning device may verify the content of the decrypted version of the client device certificate.

In one or more exemplary intraoral scanning devices, to verify the client device data comprises to determine if the authentication key identifier matches a client device key identifier of the client device certificate, and verification fails if no match is determined.

The intraoral scanning device may be configured to verify that the authentication key identifier matches a corresponding client device key identifier comprised in the client device certificate. The intraoral scanning device may be configured to verify that the authentication key identifier has a value that is equal to the client device key identifier comprised in the client device certificate. For example, the intraoral scanning device may be configured to verify that the authentication key identifier matches a corresponding client device key identifier comprised in the decrypted version of the client device certificate. In one or more exemplary intraoral scanning devices, to verify the client device data comprises to determine if a client device type identifier of the client device certificate is valid and verification fails if the client device type identifier of the client device certificate is not valid. For example, the intraoral scanning device may be configured to verify that the authentication type identifier matches a corresponding client device type identifier comprised in the decrypted version of the client device certificate.

In one or more exemplary intraoral scanning devices, to determine if a client device type identifier of the client device certificate is valid comprises to determine if the client device type identifier is black-listed, wherein the client device type is not valid if the client device type identifier is black-listed, e.g. appears on a list of black-listed client device types. In one or more exemplary intraoral scanning devices, to determine if a client device type identifier of the client device certificate is valid comprises to determine if the client device type identifier is allowed, wherein the client device type is valid if the client device type identifier is allowed, e.g. appears on a list of allowed client device types. For example, the client device type identifier of the client device may be valid if the authentication type identifier matches a corresponding client device type identifier comprised in the decrypted version of the client device certificate.

In one or more exemplary intraoral scanning devices, to verify the client device data comprises to verify a digital signature of the client device certificate, and verification fails if the digital signature is not verified. For example, the client device data comprises a digital signature appended to it to protect integrity of the client device data. Verifying a digital signature comprises e.g. computing a comparison result based on the digital signature and a corresponding client device public key and comparing the comparison result to the received client device data/client device certificate. The corresponding client device public key may be retrieved by the intraoral scanning device from the memory unit, a remote data storage unit, and/or the server device. The digital signature may be verified as valid, or the verification is successful when the digital signature raised to the power of the client device public key is identical to the received client device data.

In one or more exemplary intraoral scanning devices, the client device certificate comprises a signing device identifier and/or a client device identifier. The client device identifier refers to an identifier identifying a client device. The client device identifier may for example comprise a medium access control, MAC, address of the client device, and/or a serial number of the client device. The intraoral scanning device may be configured to verify the client device data by determining if the signing device identifier and/or the client device identifier are valid. For example, the intraoral scanning device may be configured to determine if the signing device identifier is valid by verifying that the signing device identifier is not black-listed. For example, the intraoral scanning device may be configured to determine if the client device identifier is valid by verifying that the client device identifier is not black-listed. The client device identifier allows for example the intraoral scanning device to identify the client device amongst a plurality of client devices. Verification fails if the signing device identifier and/or the client device identifier are not valid. For example, if the intraoral scanning device determines that the signing device identifier and/or the client device identifier are black-listed, the signing device identifier and/or the client device identifier are not valid and verification fails.

In one or more exemplary intraoral scanning devices, the processing unit may be configured to receive an additional authentication message. The additional authentication message may comprise client device data and/or an authentication device identifier. The authentication device identifier may refer to an identifier enabling authentication of the client device, such as a client device identifier comprised in an authentication message. For example, the authentication device identifier comprises a serial number, a medium access control, MAC, address, or any combination thereof. The intraoral scanning device may be configured to verify the authentication message and authenticate the client device sending the authentication message. The processing unit may be configured to obtain a common secret based on the authentication device identifier from the memory unit. The memory unit may have client device identifiers associated with common secrets stored thereon. The processing unit may then be configured to retrieve the corresponding common secret based on the authentication device identifier. The common secret has been generated and stored earlier at e.g. an initial round of authentication of a returning client device. Thus, once the client device authenticated, the processing unit can just retrieve the corresponding common secret. This provides a faster subsequent authentication, and avoids having to regenerate the common secret for computing the additional certificate key, and thus saves the corresponding power consumption. The processing unit may be configured to generate an additional certificate key from the common secret; and to verify the client device data based on the additional certificate key. For example, the processing unit may generate the additional certificate key by computing a hash value based on the common secret and a certificate value. As described above, the processing unit may be configured to verify the client device data based on the additional certificate key by verifying the integrity of the client device data, such as verifying a MAC and/or a digital signature of the client device data. The processing unit is configured to verify the client device data based on the additional certificate key by decrypting the client device data using the additional certificate key (as a decryption key), when the client device data is received encrypted. The processing unit is configured to verify the client device data by verifying the content of the client device data. The processing unit may be configured to verify the client device data based on the additional certificate key by comparing the client device data with data stored in the memory unit.

In one or more exemplary intraoral scanning devices, the processing unit may be configured to generate an offline session key based on the common secret and the session identifier, and the processing unit may be configured to communicate with the client device using the offline session key. An offline session key may be used to secure offline communication between the intraoral scanning device and a client device. Offline communication refers to a communication that does not involve any other network device (e.g. a server device). To generate an offline session key may comprise to generate an offline key based on the common secret (e.g. perform a hash function of the common secret and an offline value), and to compute the offline session key based on the offline key and the session identifier (e.g. perform a hash function of the offline key and the session identifier). The offline session key is used by the intraoral scanning device and the client device to secure (e.g. encrypt) the intraoral scanning device data communicated between the intraoral scanning device and the client device.

In one or more exemplary intraoral scanning devices, the authentication message comprises an authentication token identifier, and the processing unit may be configured to store the authentication token identifier in the memory unit and to link the authentication token identifier with the common secret. The authentication token identifier may be indicative of enabling a token-based authentication at the intraoral scanning device, i.e. when the intraoral scanning device receives an authentication token identifier from an authenticated client device, it may enable token-based authentication in future communication with the same client device by storing e.g. an indicator such as a flag in relation with the common secret and the client. For example, the intraoral scanning device receiving the authentication token identifier may be configured to indicate to the processing unit to enable token-based authentication by storing and/or linking the token identifier with the common secret generated for the same client device, such as by storing and/or linking the token identifier with the common secret and the client device identifier of the same client device in e.g. a table. Token identifiers and token-based authentication may be used for intraoral scanning device management, such as to group intraoral scanning devices within a dental clinic, and permit further customization with minimal or no user physical interaction/intervention as well as possibly simpler and faster client device authentication. The client device for example accesses securely a data storage where the token identifier is securely stored in a first session, retrieves the credential and keying material to perform token-based authentication in a subsequent session. This way, any client device in e.g. a dental clinic can be used to perform updates of the intraoral scanning device in a secure way using token-based authentication.

In one or more exemplary intraoral scanning devices, the processing unit may be configured to receive a further authentication message comprising client device data, an authentication type identifier, an authentication key identifier and/or an authentication session token identifier. The further authentication message may comprise an authentication device identifier. The processing unit may be configured to find in the memory unit the common secret linked to the client device type identifier and/or the client device identifier of the client device that sends the further authentication message based on locating the stored client device type identifier corresponding to the authentication type identifier and/or locating the stored client device identifier corresponding to the authentication device identifier. The processing unit may be configured to obtain a common secret based on the authentication type identifier; to generate a token key based on the common secret; and to generate a session token identifier based on the token key and the session identifier. The processing unit may have generated in an earlier session with the client device a common secret to e.g. establish a certificate key and may have stored and linked the common secret to the client device type identifier and/or the client device identifier. The processing unit may then be configured to obtain the common secret based on the authentication type identifier and/or the authentication client identifier corresponding to the stored client device type identifier and/or client device identifier. The processing unit may be configured to generate a token key by performing a hash function on the common secret and a token value (such as a pre-defined arbitrary string or a pre-defined arbitrary value). The processing unit may be configured to generate a session token identifier based on the token key and the session identifier by generating a session identifier, and by performing a hash function on the token key and the session identifier. The processing unit may be configured to verify the authentication session token identifier based on the session token identifier. The processing unit may be configured to verify the authentication session token identifier by comparing the authentication session token identifier and the generated session token identifier. For example, if the processing unit determines that the authentication session token identifier matches the generated session token identifier, the verification is successful and the processing unit may proceed with no user physical intervention, and continue to verify the client device data provided in the further authentication message. The client device data may comprise a client device certificate. The intraoral scanning device may verify the client device certificate (and/or check against a blacklist) for any customization or updates to be allowed. The verified authentication token identifier may for example be used to indicate to the intraoral scanning device that the client device holds the previous shared token key and therefore is allowed to customize exactly this intraoral scanning device without physical intraoral scanning device user intervention.

In one or more exemplary intraoral scanning devices, the processing unit may be configured to generate a session key based on the session identifier and the intraoral scanning device key, and the processing unit may be configured to receive and authenticate session data based on the session key. To generate a session key based on the session identifier and the intraoral scanning device key may comprise computing the session key by generating a common secret based on the intraoral scanning device key and the session identifier and optionally generating a hash value of the common secret and a session value, the generated hash value corresponding to the session key. For example, the processing unit may be configured to authenticate session data based on the session key by verifying a MAC generated with the session key and/or by decrypting session data using the session key. The present disclosure relates to a method of operating an intraoral scanning device comprising a processing unit configured to process intraoral scan data of a patient and provide 2D image data and/or 3D image data, a memory unit, and a wireless interface, such as a method for controlling communication of an intraoral scanning device, such as a method for enabling secure intraoral scanning device communication. The method comprises receiving a linking request for a session via the wireless interface. The linking request may comprise an authentication key identifier and/or an authentication type identifier, in order to permit the intraoral scanning device to perform authentication at this early stage the linking request and the client device sending the linking request. This may provide a level of access control. The method comprises obtaining a session identifier, e.g. with the intraoral scanning device. Obtaining a session identifier may comprise generating a session identifier, such as by generating a random or pseudo-random number. For example, the processing unit generates a random or pseudo-random number of a predetermined length, e.g. 16 bytes, 32 bytes, 64 bytes etc., to be used as a session identifier. Obtaining a session identifier may comprise retrieving a session identifier from the memory unit. The method may comprise storing the session identifier in the memory unit. For example, storing the session identifier in the memory unit comprises storing the session identifier at a memory address of the memory unit, and/or in memory cells of the memory unit, such as in designated memory cells and/or at designated addresses.

The method comprises transmitting via the wireless interface a linking response comprising an intraoral scanning device identifier and the session identifier. Transmitting the linking response may comprise generating the linking response by including the session identifier and the intraoral scanning device identifier and transmitting the thus generated linking response to e.g. the client device.

The method comprises receiving, via the wireless interface, an authentication message. The authentication message comprises an authentication key identifier and client device data. The method may comprise receiving, via the wireless interface, an authentication message from a client device. For example, the intraoral scanning device receives the authentication message from the client device in order to establish a communication session. The client device data may comprise a client device certificate, customization data, intraoral scanning device operating parameters, and/or firmware data. The authentication key identifier may be an identifier that may be used to verify if the client device provides an authentication key identifier acceptable by the intraoral scanning device.

The method comprises selecting an intraoral scanning device key from a plurality of intraoral scanning device keys in the memory unit, based on the authentication key identifier. When the authentication key identifier matches the intraoral scanning device key identifier held by the intraoral scanning device and/or is indicative of an intraoral scanning device key of the intraoral scanning device, the processing unit may be configured to use the authentication key identifier as a key identifier indicating which intraoral scanning device key is to be used as keying material in the session. Selecting an intraoral scanning device key from a plurality of intraoral scanning device keys in the memory unit may be based on the authentication key identifier and an authentication type identifier. The authentication type identifier may be received in plaintext by the intraoral scanning device, and/or as client device type identifier in the client certificate (encrypted or decrypted). For example, the processing unit selects an intraoral scanning device key which the authentication key identifier and the authentication type identifier indicate.

The method comprises verifying the client device data, based on the selected intraoral scanning device key. Verifying the client device data may be based on an intraoral scanning device certificate or at least parts thereof. Further, the method comprises terminating the session if verification fails. Verifying the client device data based on the selected intraoral scanning device key may comprise verifying the integrity of the client device data based on the selected intraoral scanning device key, such as verifying a MAC and/or a digital signature comprised in the client device data. Verifying the client device data based on the selected intraoral scanning device key may comprise decrypting the client device data using the selected intraoral scanning device key (as keying material to derive a decryption key or as a decryption key), when the client device data is received encrypted. Verifying the client device data based on the selected intraoral scanning device key may comprise verifying the client device data by comparing the received client device data, e.g. decrypted client device certificate, with data stored in the memory unit. For example, verification fails if integrity of the client device data is detected as corrupted by e.g. verifying a MAC or a digital signature, if decryption fails, and/or if comparison of the received client device data with data stored in the memory unit shows a mismatch.

The authentication message optionally comprises an authentication type identifier. An authentication type identifier may be indicative of a client device type identifier and/or a certificate type identifier. Selecting an intraoral scanning device key from a plurality of intraoral scanning device keys may be based on the authentication type identifier. Selecting the intraoral scanning device key may be based on the authentication type identifier provided in the authentication message and/or the authentication key identifier verified.

The client device data may comprise a client device certificate (such as an encrypted client device certificate), an authentication key identifier, and/or an authentication type identifier. The client device may be assigned a client device certificate.

The method may comprise generating a certificate key based on the selected intraoral scanning device key and/or the session identifier; and verifying the client device data may comprise decrypting the encrypted client device certificate with the certificate key to obtain a decrypted version of the encrypted client device certificate. Decrypting the encrypted client device certificate with the certificate key may comprise decrypting the encrypted client device certificate using a certificate key, a common secret and/or an intraoral scanning device key, such as generating a certificate key based on a common secret, and processing the encrypted client certificate using a decryption function and a certificate key. The certificate key may be based on a common secret and/or a certificate value. Generating a certificate key may comprise obtaining or generating the common secret based on the selected intraoral scanning device key. For example, generating the common secret based on the intraoral scanning device key comprises retrieving the intraoral scanning device certificate from the memory unit, the intraoral scanning device certificate comprising the selected intraoral scanning device key, and/or retrieving the selected intraoral scanning device key from the memory unit. The method may comprise generating the common secret based on a session identifier and/or the intraoral scanning device key. For example, the common secret CS is generated based on a selected intraoral scanning device key and a session identifier, e.g. as follows:

$$CS = hash ( HD\_KEY , S\_ID ) ,$$

where hash is a hash function, HD_KEY is the selected intraoral scanning device key and S_ID is a session identifier. The session identifier may comprise a random or pseudo random number of a defined length. The common secret may be used as a certificate key in one or more exemplary intraoral scanning devices. The intraoral scanning device may be configured to store the common secret in the memory unit, so as to e.g. retrieve the common secret from the memory unit when needed.

Generating a certificate key may comprise performing a hash function on the common secret and/or a certificate value. The intraoral scanning device may then generate the certificate key e.g. as follows:

$$C\_KEY = hash ( CS , C\_VAL ) ,$$

where hash is a hash function, CS is the common secret and C_VAL is a certificate value. The certificate value may be a predefined value or string, such as “certificate”.

In one or more exemplary methods, generating a certificate key comprises performing a hash function on the intraoral scanning device key and the session identifier. Stated differently, the common secret may be used as a certificate key if the client device has also used the common secret as certificate key to encrypt the client device certificate.

Verifying the client device data may comprise decrypting the encrypted client device certificate using the certificate key generated by the intraoral scanning device and obtaining the decrypted version of the client device certificate.

In one or more exemplary methods, verifying the client device data may comprise verifying a content of the decrypted version of the client device certificate. For example, verifying the client device data comprises determining if the authentication key identifier matches a client device key identifier of the client device certificate, and verification fails if no match is determined.

In one or more exemplary methods, verifying the client device data comprises determining if a client device type identifier of the client device certificate is valid and verification fails if the client device type identifier of the client device is not valid. For example, an authentication type identifier is sent in plain text in the authentication message, the authentication type identifier sent in plain text is valid if the authentication type identifier matches a corresponding client device type identifier comprised in the decrypted version of the client device certificate. For example, determining if a client device type identifier of the client device certificate is valid may comprise determining if the client device type identifier of the client device certificate is comprised in a list of authorized client devices.

In one or more exemplary methods, determining if a client device type identifier of the client device certificate is valid comprises determining if the client device type identifier is black-listed, wherein the client device type is not valid if the client device type identifier is black-listed, e.g. appears on a list of black-listed client device types. In one or more exemplary methods, determining if a client device type identifier of the client device certificate is valid comprises determining if the client device type identifier is allowed, wherein the client device type is valid if the client device type identifier is allowed, e.g. appears on a list of allowed or authorized client device types.

In one or more exemplary methods, verifying the client device data comprises verifying a digital signature of the client device certificate, and verification fails if the digital signature is not verified. For example, the client device data comprises a digital signature appended to it to protect integrity of the client device data. Verifying a digital signature comprises e.g. computing a comparison result based on the digital signature and a corresponding public key and comparing the comparison result to the received client device data. The digital signature may be verified as valid, or the verification may be successful when the digital signature raised to the power of the public key is identical to the received client device data.

In one or more exemplary methods, the client device certificate comprises a signing device identifier and/or a client device identifier, and verifying the client device data comprises determining if the signing device identifier and/or the client device identifier is valid and wherein verification fails if the client device identifier of the client device and/or the signing device identifier is not valid.

In one or more exemplary methods, determining if a client device identifier of the client device certificate is valid comprises determining if the client device identifier is black-listed, wherein the client device identifier is not valid if the client device identifier is black-listed, e.g. appears on a list of black-listed client devices. In one or more exemplary methods, determining if a client device identifier of the client device certificate is valid comprises determining if the client device identifier is allowed, wherein the client device type is valid if the client device identifier is allowed, e.g. appears on a list of allowed or authorized client devices.

In one or more exemplary methods, the method comprises receiving an additional authentication message comprising client device data and/or an authentication device identifier. The method may further comprise obtaining, from the memory unit, a common secret based on the authentication device identifier, generating an additional certificate key from the common secret, and verifying the client device data based on the additional certificate key.

In one or more exemplary methods, the method comprises generating an offline session key based on the common secret and the session identifier, and communicating with the client device using the offline session key.

In one or more exemplary methods, the method comprises receiving a further authentication message comprising client device data, an authentication type identifier, an authentication key identifier and/or an authentication session token identifier. The further authentication message may comprise an authentication device identifier. The method may comprise finding or determining in the memory unit the common secret linked to the client device type identifier and/or the client device identifier of the client device that sends the further authentication message based on locating the stored client device type identifier corresponding to the authentication type identifier and/or locating the stored client device identifier corresponding to the authentication device identifier. The method may comprise obtaining a common secret based on the authentication type identifier; generating a token key based on the common secret; and generating a session token identifier based on the token key and the session identifier. The processing unit may have generated in an earlier session with the client device a common secret to e.g. establish a certificate key and may have stored and linked the common secret to the client device type identifier and/or the client device identifier. The method may comprise obtaining the common secret based on the authentication type identifier and/or the authentication client identifier corresponding to the stored client device type identifier and/or client device identifier. The method may comprise generating a token key by performing a hash function on the common secret and a token value (such as a pre-defined arbitrary string or a pre-defined arbitrary value). The method may comprise generating a session token identifier based on the token key and the session identifier by generating a session identifier, and by performing a hash function on the token key and the session identifier. The method may comprise verifying the authentication session token identifier based on the session token identifier. The method may comprise verifying the authentication session token identifier by comparing the authentication session token identifier and the generated session token identifier. For example, if it is determined that the authentication session token identifier matches the generated session token identifier, the verification is successful and the processing unit may proceed with no user physical intervention, and continue to verify the client device data provided in the further authentication message. The client device data may comprise a client device certificate. The intraoral scanning device may verify the client device certificate (and check against a blacklist) for any customization to be allowed. The verified authentication token identifier may for example be used to indicate to the intraoral scanning device that the client device holds the previous shared token key and therefore is allowed to customize exactly this intraoral scanning device without physical intraoral scanning device user intervention.

In one or more exemplary methods, the method comprises generating a session key based on the session identifier and the intraoral scanning device key, receiving and authenticating session data based on the session key.

Client Device with Certificate and Related Method:

An aspect of the present disclosure to provide a client device, and a method which seeks to mitigate, alleviate, or eliminate one or more of the above-identified deficiencies in the art and disadvantages singly or in any combination.

A further aspect of the present disclosure is to improve security in wireless communication with an intraoral scanning device that protects the intraoral scanning device against potential attacks, such as an improved client device, and a method of communication with an intraoral scanning device that improves security thereof.

There is a need for client device and method providing improved security for intraoral scanning device communication. Further, there is a need for devices and methods reducing the risk of an intraoral scanning and intraoral scanning function being compromised by a third party.

According to the aspects, a client device for intraoral scanning device communication is disclosed. The client device may comprise a processing unit, a memory unit and a wireless interface. The memory unit may have a client device key, such as at least one client device key, and/or a client device certificate stored thereon. The processing unit may be configured to receive a connection response, e.g. comprising an intraoral scanning device identifier, via the wireless interface; generate one or more keys, e.g. including a certificate key, based on the intraoral scanning device identifier and/or the client device key; obtain an authentication message based on the certificate key and/or the client device certificate. To obtain the authentication message may optionally comprise to generate and/or obtain an encrypted client device certificate by encrypting the client device certificate, e.g. with the certificate key, and optionally to include the encrypted client device certificate in the authentication message. The processing unit may be configured to transmit the authentication message via the wireless interface.

The processing unit may be configured to obtain a session identifier. To generate one or more keys may comprise to generate an intraoral scanning device key based on the intraoral scanning device identifier and the client device key, and may further comprise to generate a common secret based on the intraoral scanning device key and the session identifier.

The certificate key may be based on the common secret and a certificate value.

To generate one or more keys may comprise to generate a session key based on the intraoral scanning device identifier, the session identifier and the client device key. The processing unit may be configured to transmit the session key to a customization device.

The session key may be based on the common secret and a session value.

The processing unit may further be configured to include an authentication key identifier indicative of the client device key in the authentication message.

The processing unit may further be configured to include an authentication type identifier in the authentication message.

The client device certificate may comprise one or more of.
a certificate type identifier;
a signing device identifier;
a client device type identifier;
a client device identifier;
a client device key identifier;
one or more hardware identifiers; and
a digital signature.

According to the aspects, a method of operating a client device for intraoral scanning device communication is disclosed, the client device comprising a memory unit having a client device key, such as at least one client device key, and/or a client device certificate stored thereon. The method comprises receiving a connection response, e.g. comprising an intraoral scanning device identifier via the wireless interface; generating one or more keys, e.g. including a certificate key, based on the intraoral scanning device identifier and/or the client device key; and obtaining an authentication message based on the certificate key and/or the client device certificate. Obtaining the authentication message optionally comprises generating an encrypted client device certificate, e.g. by encrypting the client device certificate with the certificate key, and optionally including the encrypted client device certificate in the authentication message. The method comprises transmitting the authentication message via the wireless interface.

The method may comprise obtaining a session identifier, and wherein generating one or more keys may comprise generating an intraoral scanning device key based on the intraoral scanning device identifier and the client device key, and generating a common secret based on the intraoral scanning device key and the session identifier.

The certificate key may be based on the common secret and a certificate value, or the method may comprise basing the certificate key on the common secret and a certificate value.

Generating one or more keys may comprise generating a session key based on the intraoral scanning device identifier, the session identifier and the client device key. The method may further comprise transmitting the session key to a customization device.

The session key may be based on the common secret and a session value, or the method may comprise basing the session key on the common secret and a session value.

Obtaining the authentication message may comprise including an authentication key identifier indicative of the client device key in the authentication message.

Obtaining the authentication message may comprise including an authentication type identifier in the authentication message.

Advantageously, the method and intraoral scanning device enables the intraoral scanning device manufacturer to control client device access to the intraoral scanning device and/or enable version control in client device access.

The method and apparatus as disclosed provide a scalable security architecture for intraoral scanning device systems with improved security. The disclosed client device and method support an intraoral scanning device in combatting attacks such as unauthorized access or control of an intraoral scanning device, while still allowing access to legitimate parties such as the client device, for e.g. customization purposes, update purposes, maintenance purposes. The client device and method allow the intraoral scanning device to open a session only with authenticated parties, such as an authenticated customization device, an authenticated accessory device, an authenticated external device and/or an authenticated server. This may provide robustness against impersonation and masquerading attacks, battery exhaustion attacks, man-in-the-middle attacks and/or replay attacks. Further, the need for updating and/or exchange of keys in case a key has been compromised at a client device has been reduced and simplified.

The processing unit may be configured to process the received 2D image data and/or 3D image data for the purpose of visualizing the data on a display, for designing dental accessories, such as aligners, retainers, crowns, implants, bracers, nightguards etc, and/or for providing diagnostic data.

The 2D image data and/or the 3D image data may be image data configured to be visualizable on a display in a 2D or a 3D manner, respectively.

The term “client device” as used herein refers to a device that communicates with the intraoral scanning device. The client device may refer to a computing device acting as a client. The client device may comprise a customization device, a handheld device, a relay, a tablet, a personal computer, a mobile phone, and/or USB dongle plugged into a personal computer. The client device may control operation of the intraoral scanning device, either by sending customization data, intraoral scanning device operating parameters, and/or firmware data. The disclosed client device and method support the intraoral scanning device in combatting attacks such as unauthorized access or control of an intraoral scanning device, while still allowing access to legitimate parties such as the client device, for e.g. customization purposes, update purposes, maintenance purposes.

The intraoral scanning device may be operated in one or more modes. The one or more modes may include a first mode and/or a second mode. The one or more modes may include a third mode and/or a fourth mode. The one or more modes may include a default mode.

The client device may comprise a memory unit and a wireless interface respectively connected to the processing unit. The wireless interface may comprise a wireless transceiver, e.g. configured for wireless communication at frequencies in the range from 2.4 to 2.5 GHz, 2.4 GHz to 5 GHz, about 2.45 GHz or about 5 GHz. The wireless transceiver may be a Bluetooth transceiver, a Bluetooth Low Energy transceiver, or a Wireless Fidelity (WIFI) transceiver. The wireless interface may form a connection to one or more other devices such as a computer, and/or a scan computer, and/or a tablet and/or a smart phone.

In an embodiment, the wireless interface is configured for communication, such as wireless communication, with an intraoral scanning device comprising a wireless transceiver.

The processing unit may be configured to send a session request for a session to the intraoral scanning device via the wireless interface. The processing unit may be configured to receive a session response from the intraoral scanning device via the wireless interface, e.g. from an intraoral scanning device and/or a session key apparatus. The session response may comprise the intraoral scanning device identifier or an identifier derived therefrom. In an exemplary client device, the client device may receive the intraoral scanning device identifier during a pairing of the client device and the intraoral scanning device. Hence, the processing unit comprises e.g. a receive/send unit configured to send data such as the session request and/or receive data such as the session response via the wireless interface. The processing unit may be configured to obtain a session key based on e.g. the session response, such as to extract the session key from or based on the session response. Hence, the processing unit comprises an obtainer. The processing unit may retrieve the session key from a key depository, e.g. stored in the memory unit. The processing unit may be configured to obtain a session key, wherein to obtain a session key may comprise to establish a connection to a session key apparatus via the wireless interface. The processing unit may send a session key request to the session key apparatus such as a session key server via the wireless interface e.g. via a wireless communication link established between the client device and the session key apparatus via the wireless interface. The processing unit may receive a session key response from the session key apparatus via the wireless interface, and may determine the session key based on the session key response.

The session response may comprise an intraoral scanning device identifier. The intraoral scanning device identifier may comprise a hardware number of the intraoral scanning device and/or a serial number of the intraoral scanning device. The client device may retrieve the session key from the session key apparatus by providing the intraoral scanning device identifier to the session key apparatus, e.g. as part of the session key request, and requesting the session key or an intraoral scanning device key from the session key apparatus and/or requesting the session key apparatus to decrypt the session response and/or the session key.

In one or more exemplary client devices, the processing unit configured to obtain the session key may be configured to establish a connection to a session key apparatus via the wireless interface, to send a session key request to the session key apparatus via the wireless interface, to receive a session key response from the session key apparatus via the wireless interface, and to determine the session key based on the session key response. The session key request may comprise the intraoral scanning device identifier. The connection to the session key apparatus may be a secure connection over a network, such as including a private and/or a public network.

The session key apparatus may be a customization accessory device; wherein the customization accessory device optionally comprises a storage device containing a list configured to provide a session key and/or a session key response based on a session key request.

The processing unit may be configured to determine intraoral scanning device data. Hence the processing comprises e.g. a determiner. The intraoral scanning device data comprises e.g. firmware, customization data, and/or intraoral scanning device operating parameters. Customization data may for example be setting data of the intraoral scanning device, such as power management settings, configuration settings, configuration of a user interface of the intraoral scanning device and/or settings of an optical unit of the intraoral scanning device. Firmware may refer to a computer program provided by the intraoral scanning device manufacturer, and to be installed on the intraoral scanning device to control the intraoral scanning device. Firmware is for example to be installed to upgrade the operations and capabilities of the intraoral scanning device.

The optical unit may include one or more light projectors, one or more optical components, and one or more image sensors.

The user interface of the intraoral scanning device may include at least a touch sensor, at least a touch button, at least a light emitting diode, a haptic sensor, and/or an accelerometer.

The session response may comprise an encrypted session key. The processing unit may be configured to determine the session key by retrieving the session key from the session key response.

In one or more exemplary client device, to determine the session key comprises retrieving an intraoral scanning device key from the session key response or from the memory unit and decrypting the encrypted session key based on the intraoral scanning device key. To determine the session key may comprise decrypting the encrypted session key with a global key. A global is e.g. a key common to a group of client devices. The processing unit may be configured to retrieve an intraoral scanning device key from the session key response and decrypt the encrypted session key based on the intraoral scanning device key. The processing unit may comprise a decrypt/encrypt unit. The intraoral scanning device key may be e.g. a symmetric key or a public key of a private-public key pair. The intraoral scanning device key may comprise an AES-128 bits key as a symmetric key. The use of a symmetric key as an intraoral scanning device key provides the advantage of being able to use hardware accelerators. The intraoral scanning device key may comprise a public key of a private-public key pair, such as a public key of a private-public key pair of an authorized discloser of the session key, such as of the client device or the session key apparatus.

The processing unit may be configured to determine the session key by including a decryption of the encrypted session key with a global key, i.e. to determine the session key may comprise decrypting the encrypted session key with a global key. The global key may be e.g. a symmetric key or a public key of a private-public key pair. The session key may be compliant with an encryption standard such as Advanced Encryption Standard, AES, RSA crypto-system, Triple Data Encryption Algorithm.

The processing unit may be configured to generate session data, e.g. including a message authentication code, based on the session key and the intraoral scanning device data. Hence the processing unit may comprise a generator. The processing unit may generate a message authentication code based on the session key and the intraoral scanning device data. The message authentication code may be included in the session data. The processing unit may be configured to generate session data based on an intraoral scanning device key. The processing unit may be configured to digitally sign the intraoral scanning device data, such as to digitally sign the intraoral scanning device data using a private key of the client device, and/or of a group of client devices. The processing unit may be configured to digitally sign the intraoral scanning device data using a private key obtained from the session key apparatus, e.g. as part of a session key response. The processing unit may generate a digital signature using a signature generation function and a private key of a client device and append the digital signature to the session data. The intraoral scanning device may then verify the digital signature when receiving the session data. If the digital signature is not successfully verified using the alleged public key of a client device, the intraoral scanning device may disregard the session data and/or terminate the session. This may provide the advantage that the client device supports the intraoral scanning device in rejecting session data tampered or received from unauthenticated parties and the communication with the intraoral scanning device may thus be robust against impersonation and masquerading attacks.

The processing unit may be configured to send the session data to the intraoral scanning device via the wireless interface, e.g. using the receive/send unit. The session data may comprise intraoral scanning device data encrypted with the session key. To encrypt session data with the session key, the client device may utilize any of the above encryption standards.

The present disclosure relates to improved security in intraoral scanning device communication.—Namely, the client device disclosed herein enables intraoral scanning device communication that is robust against security threats, vulnerabilities and attacks by implementing appropriate safeguards and countermeasures, such as security mechanisms, to protect against threats and attacks. The present disclosure relates to intraoral scanning device communication that is robust against replay attacks, unauthorized access, battery exhaustion attacks, and man-in-the-middle attacks.

As used herein, the term “intraoral scanning device” refers to a device configured to acquire intraoral scan data from a three-dimensional dental object during a scanning session.

As used herein, the term “certificate” refers to a data structure that enables verification of its origin and content, such as verifying the legitimacy and/or authenticity of its origin and content. The certificate is configured to provide a content that is associated to a holder of the certificate by an issuer of the certificate. The certificate comprises a digital signature, so that a recipient of the certificate is able to verify or authenticate the certificate content and origin. The certificate may comprise one or more identifiers and/or keying material, such as one or more cryptographic keys (e.g. an intraoral scanning device key) enabling secure communication in an intraoral scanning device system. The certificate permits thus to achieve authentication of origin and content, non-repudiation, and/or integrity protection. The certificate may further comprise a validity period, one or more algorithm parameters, and/or an issuer. A certificate may comprise a digital certificate, a public key certificate, an attribute certificate, and/or an authorization certificate. Examples of certificates are X.509 certificates, and Secure/Multipurpose Internet Mail Extensions, S/MIME, certificates, and/or Transport Layer Security, TLS, certificates.

As used herein, the term “key” refers to a cryptographic key, i.e. a piece of data, (e.g. a string, a parameter) that determines a functional output of a cryptographic algorithm. For example, during encryption, the key allows a transformation of a plaintext into a cipher-text and vice versa during decryption. The key may also be used to verify a digital signature and/or a message authentication code, MAC. A key is so called a symmetric key when the same key is used for both encryption and decryption. In asymmetric cryptography or public key cryptography, a keying material is a key pair, so called a private-public key pair comprising a public key and a private key. In an asymmetric or public key cryptosystem (such as Rivest Shamir Adelman, RSA, cryptosystem), the public key is used for encryption and/or signature verification while the private key is used for decryption and/or signature generation. The intraoral scanning device key may be keying material allowing derivation of one or more symmetric keys, such as a session key and/or a certificate key for intraoral scanning device communication. The intraoral scanning device key may be stored in a memory unit of the intraoral scanning device, e.g. during manufacture. The intraoral scanning device key may comprise keying material that is used to derive a symmetric key. The intraoral scanning device key comprises for example an Advanced Encryption Standard, AES, key, such as an AES-128 bits key.

As used herein the term “identifier” refers to a piece of data that is used for identifying, such as for categorizing, and/or uniquely identifying. The identifier may be in a form of a word, a number, a letter, a symbol, a list, an array or any combination thereof. For example, the identifier as a number may be in the form of an integer, such as unsigned integer, unit, with a length of e.g. 8 bits, 16 bits, 32 bits, etc., such as an array of unsigned integers.

A client device for intraoral scanning device communication with an intraoral scanning device is disclosed. The term “client device” as used herein refers to a device that is able to communicate with the intraoral scanning device. The client device may refer to a computing device acting as a client. The client device may comprise a customization device, a handheld device, a relay, a tablet, a personal computer, an application running on a personal computer or tablet, or mobile phone and/or USB dongle plugged into a personal computer. The client device may be attributed a client device type indicated by a client device type identifier, the client device type e.g. corresponding to a model, category or type of client devices, such as a customization type, e.g. a tablet product model, category or type for customizing the intraoral scanning device, a USB dongle product model, category or type for customizing the intraoral scanning device. The client device may be configured to control operation of the intraoral scanning device, either by sending customization data, intraoral scanning device operating parameters, and/or firmware data.

The client device comprises a memory unit and a wireless interface respectively connected to the processing unit. The memory unit may include removable and non-removable data storage units including, but not limited to, Read Only Memory (ROM), Random Access Memory (RAM), etc. The memory unit has a client device certificate stored thereon. The memory unit may have the client device certificate and/or the client device key stored at a memory address of the memory unit, and/or in memory cells of the memory unit, such as in designated memory cells and/or at designated addresses. The wireless interface may comprise a wireless transceiver, e.g. configured for wireless communication at frequencies in the range from 2.4 to 2.5 GHz. The wireless interface may comprise one or more connectors for connection to another device, e.g. a customization device. A connector may be a standard connector, such as a USB connector (USB 2.0 standard-A, USB 2.0 standard-B, Micro-A USB, Micro-B USB, Mini-A USB, Mini-B USB or others). A connector may be a proprietary connector used by a manufacturer of personal electronic devices. The wireless interface may be configured for communication, such as wireless communication, with an intraoral scanning device comprising a wireless transceiver. The client device certificate may comprise a certificate type identifier. The certificate type identifier may indicate a type of the certificate amongst a variety of certificate types, such as an intraoral scanning device family certificate type, an intraoral scanning device certificate type, a firmware certificate type, a research and development certificate type, and/or a client device certificate type. The certificate type identifier may be used by an intraoral scanning device and/or the client device to identify what type of certificate an intraoral scanning device receives, stores, authenticates and/or retrieves. The client device certificate may comprise a version identifier indicative of a data format version of the certificate. An intraoral scanning device may use the certificate type identifier and/or the version identifier of the client device certificate to determine what type of data the client device certificate comprises and/or what type of data is comprised in a field of the client device certificate. For example, an intraoral scanning device may determine based on the certificate type identifier and/or version identifier what field of the client device certificate comprises a digital signature and/or which public key is needed to verify the digital signature of the client device certificate. It may be envisaged that there is a one-to-one mapping between the certificate type identifier and the public-private key pair.

The client device certificate may comprise a signing device identifier. The signing device identifier refers to a unique identifier identifying the device that has signed the client device certificate, such as a manufacturing device, e.g. an integrated circuit card, a smart card, a hardware security module. The signing device identifier may for example comprise a medium access control, MAC, address of the signing device and/or a serial number of the signing device. The signing device identifier may allow for example an intraoral scanning device to determine whether the signing device is e.g. black-listed or not, and thus to reject certificates signed by a signing device that has been black-listed, e.g. due to theft or other corruption.

The client device certificate may comprise a client device type identifier. The client device type identifier may indicate a type of the client device amongst a variety of client device types, such as a model, category or type of client devices, a USB dongle product model, category or type for customizing the intraoral scanning device. The client device type identifier may be used by an intraoral scanning device to identify what type of client device the intraoral scanning device communicates with. The client device type identifier may enable an intraoral scanning device to select a set of keys from a plurality of key sets in the intraoral scanning device. Respective key sets in the intraoral scanning device may be used by respective different types of client devices.

The client device certificate may comprise a client device identifier. The client device identifier may be based on one or more hardware identifiers of one or more hardware components/modules of the client device.

The client device certificate may comprise a client device key identifier. The client device key identifier is indicative of the client device key.

The client device certificate may comprise one or more hardware identifiers, for example a first hardware identifier and/or a second hardware identifier. A hardware identifier may identify a piece of hardware comprised in the client device, such as a radio chip comprised in the client device or a digital signal processor of the client device. The hardware identifier(s) may be stored in a register of the piece of hardware comprised in the client device during manufacturing of the piece of hardware. The hardware identifier may comprise a serial number of the hardware, a chip identifier, or any combination thereof. The client device receiving or retrieving from the memory unit the client device certificate comprising the hardware identifier may verify the client device certificate by comparing its stored hardware identifier and the corresponding hardware identifier comprised in the client device certificate. Such verification may be performed upon retrieval of the client device certificate from the memory unit, such as at boot or power-on of the client device. The client device certificate may comprise one or more bluetooth addresses, e.g. assigned by the manufacturer during manufacture.

The client device certificate may comprise a user identifier, e.g. in the form of a user name. A client device certificate with a user identifier may facilitate the use of a generic device, such as a tablet computer, as a client device, e.g. by implementing a user verification/key generation/encryption at a remote server device, e.g. controlled by an intraoral scanning device manufacturer.

The client device certificate may comprise a digital signature. The digital signature enables a proof or verification of authenticity of the client device certificate, such as verification of the signer legitimacy. The digital signature is optionally generated by a manufacturing device using a client device family private key at manufacturing of the client device. The digital signature is verifiable by an intraoral scanning device and/or customization device using a corresponding client device family public key. If the digital signature is not successfully verified using the alleged public key, an intraoral scanning device may disregard the client device certificate and/or abort normal operation. This may provide the advantage that the intraoral scanning device rejects a client device certificate that is tampered or received from unauthenticated parties. The communication with the intraoral scanning device may thus be robust against impersonation, modification and masquerading attacks.

The processing unit is configured to receive a connection response comprising an intraoral scanning device identifier via the wireless interface. The connection response may be generated by and/or sent from an intraoral scanning device. The processing unit is configured to generate one or more keys, e.g. based on the intraoral scanning device identifier and/or the client device key. To generate one or more keys may comprise to generate a common secret based on the client device key. To generate one or more keys may comprise to generate an intraoral scanning device key based on the intraoral scanning device identifier and/or the client device key, e.g. including to perform a hash function. For example, the intraoral scanning device key, IOS_KEY, may be given as:

$$IOS\_KEY = hash ( IOS\_ID , CD\_KEY ) ,$$

where hash is a hash function, IOS_ID is the intraoral scanning device identifier and CD_KEY is the client device key.

By generating and/or using a common secret, a need for exchanging keys is avoided. Further, if the common secret is based on the intraoral scanning device identifier (client device key is different from intraoral scanning device key), the client device key cannot be derived from the intraoral scanning device key used by the intraoral scanning device. Thereby the risk of compromising the client device key is heavily reduced.

The one or more keys generated based on the intraoral scanning device identifier and/or the client device key may be based on the common secret.

The certificate key may be based on the common secret and/or a certificate value. The certificate value may be a predefined value or string, such as “certificate”. The certificate key may be generated by performing a hash function on the common secret and/or the certificate value. For example, the certificate key, C_KEY, may be given as:

$$C\_KEY = hash ( CS , C\_VAL ) ,$$

where hash is a hash function, CS is the common secret and C_VAL is the certificate value.

To generate one or more keys may comprise to generate a session key. The session may be different from the certificate key. The session key may be based on the intraoral scanning device identifier. The session key may be based on the session identifier. The session key may be based on the client device key. The processing unit may be configured to transmit the session key to a customization device. The client device, when configured to operate as a customization device, may be configured to perform customization communication with the intraoral scanning device based on the session key. The session key may be based on the common secret and/or a session value. The session value may be a predefined value or string, such as “session”. The session key may be generated by performing a hash function on the common secret and/or the session value. For example, the session key, S_KEY, may be given as:

$$S\_KEY = hash ( CS , S\_VAL ) ,$$

where hash is a hash function, CS is the common secret and S_VAL is the session value. By generating a session key based on a session identifier and a common secret, session specific communication is enabled.

The processing unit is configured to obtain an authentication message based on the certificate key and/or the client device certificate. To obtain the authentication message may comprise to include the client device certificate in the authentication message.

The processing unit may be configured to include an authentication key identifier, e.g. indicative of the client device key in the authentication message. The authentication key identifier may be indicative of or match the client device key identifier of the client device certificate. An authentication message comprising an authentication key identifier indicative of the client device key enables an intraoral scanning device to select a correct intraoral scanning device key from a plurality of intraoral scanning device keys, e.g. in order to generate or select the common secret. Subsequently, the intraoral scanning device may generate the certificate key for decrypting the encrypted client device certificate in the intraoral scanning device.

The processing unit may be configured to include an authentication type identifier in the authentication message. The authentication type identifier may be indicative of or match the client device type identifier and/or the certificate type identifier of the client device certificate. An authentication message comprising an authentication type identifier may enable an intraoral scanning device to select an intraoral scanning device key from a selected set of intraoral scanning device keys when the intraoral scanning device comprises a plurality of intraoral scanning device key sets. In addition or alternatively, the intraoral scanning device may be configured to process the authentication message in different ways based on the authentication type identifier. Thus, an intraoral scanning device may be able to select an appropriate authentication message processing scheme.

The method comprises receiving a connection response, e.g. from an intraoral scanning device, via the wireless interface. The connection response may comprise an intraoral scanning device identifier. The method comprises generating and/or obtaining one or more keys, e.g. based on the intraoral scanning device identifier and/or the client device key. Generating one or more keys may comprise to generate a common secret based on the client device key. The one or more keys may comprise a certificate key.

The method comprises generating and/or obtaining an authentication message based on the certificate key and/or the client device certificate. Obtaining and/or generating the authentication message may comprise generating an encrypted client device certificate with the client device by encrypting the client device certificate with the certificate key and optionally including the encrypted client device certificate in the authentication message. Obtaining and/or generating the authentication message may comprise obtaining an encrypted client device certificate, e.g. from the memory unit and/or a server device. Obtaining an encrypted client device certificate may comprise transmitting a certificate request to a server device. In response, the server device generates and transmits a certificate response comprising the encrypted client device certificate (the client device certificate has been encrypted with certificate key). The client device receives the certificate response with the encrypted client device certificate and includes the encrypted client device certificate in the authentication message. Thus, obtaining an encrypted client device certificate may comprise receiving a certificate response comprising the encrypted client device certificate from a server device. Obtaining the authentication message may comprise including the client device certificate in the authentication message.

The method comprises transmitting the authentication message, e.g. to the intraoral scanning device, via the wireless interface.

The method may comprise obtaining a session identifier, e.g. by receiving the session identifier from the intraoral scanning device. The connection response may comprise the session identifier. Generating one or more keys may comprise generating an intraoral scanning device key based on the intraoral scanning device identifier and the client device key. For example, the intraoral scanning device key, IOS_KEY, may be given as:

$$IOS\_KEY = hash ( IOS\_ID , CD\_KEY ) ,$$

where hash is a hash function, IOS_ID is the intraoral scanning device identifier and CD_KEY is the client device key.

Generating one or more keys may comprise generating a common secret. The common secret may be based on the intraoral scanning device key and/or the session identifier. The common secret may be based on the intraoral scanning device identifier. The intraoral scanning device key and/or the client device key may be used as a common secret. For example, the common secret, CS, may be given as:

$$CS = hash ( IOS\_KEY , S\_ID ) ,$$

where hash is a hash function, IOS_KEY is the intraoral scanning device key and S_ID is the session identifier. Generating one or more keys may comprise generating one or more keys based on the common secret.

In the method, the certificate key may be based on the common secret and/or a certificate value. The certificate value may be a predefined value or string, such as “certificate”.

Generating the certificate key may comprise performing a hash function on the common secret and/or the certificate value. For example, the certificate key, C_KEY, may be given as:

$$C\_KEY = hash ( CS , C\_VAL ) ,$$

where hash is a hash function, CS is the common secret and C_VAL is the certificate value.

Generating one or more keys may comprise generating a session key. The session key may be different from the certificate key. The session key may be based on the intraoral scanning device identifier. The session key may be based on the session identifier. The session key may be based on the client device key. The method may comprise transmitting the session key to a customization device. The method may comprise performing customization communication with the intraoral scanning device based on the session key. In the method, the session key may be based on the common secret and/or a session value.

The session value may be a predefined value or string, such as “session”. Generating the session key may comprise performing a hash function on the common secret and/or the session value. For example, the session key, S_KEY, may be given as:

$$S\_KEY = hash ( CS , S\_VAL ) ,$$

where hash is a hash function, CS is the common secret and S_VAL is the session value. By generating a session key based on a session identifier and a common secret, session specific communication is enabled.

Communication with an intraoral scanning device based on a common secret unique for the intraoral scanning device (e.g. common secret is based on intraoral scanning device identifier and/or session identifier) provides intraoral scanning device-specific communication. Thereby other intraoral scanning devices are not able to process/understand authentication messages intended for a specific intraoral scanning device.

In the method, generating the authentication message may comprise including an authentication key identifier in the authentication message. The authentication key identifier may be indicative of or match the client device key identifier of the client device certificate. An authentication message comprising an authentication key identifier indicative of the client device key enables an intraoral scanning device to select a correct intraoral scanning device key from a plurality of intraoral scanning device keys, e.g. in order to generate or select the common secret. Subsequently, the intraoral scanning device may generate the certificate key for decrypting the encrypted client device certificate in the intraoral scanning device, e.g. based on the selected intraoral scanning device key.

In the method, generating the authentication message may comprise including an authentication type identifier in the authentication message. The authentication type identifier may be indicative of or match the client device type identifier and/or the certificate type identifier of the client device certificate. An authentication message comprising an authentication type identifier may enable an intraoral scanning device to select an intraoral scanning device key from a selected set of intraoral scanning device keys when the intraoral scanning device comprises a plurality of intraoral scanning device key sets. In addition or alternatively, the intraoral scanning device may be configured to process the authentication message in different ways based on the authentication type identifier. Thus, an intraoral scanning device may be able to select an appropriate authentication message processing scheme based on the authentication type identifier. The authentication type identifier may be the client device type identifier of the client device certificate.

In an exemplary method or an exemplary client device, the common secret, CS, may be given as:

$$CS = hash ( CD\_KEY , S\_ID ) ,$$

where hash is a hash function, CD_KEY is the client device key and SID is the session identifier.

### BRIEF DESCRIPTION OF THE FIGURES

Aspects of the disclosure may be best understood from the following detailed description taken in conjunction with the accompanying figures. The figures are schematic and simplified for clarity, and they just show details to improve the understanding of the claims, while other details are left out. Throughout, the same reference numerals are used for identical or corresponding parts. The individual features of each aspect may each be combined with any or all features of the other aspects. These and other aspects, features and/or technical effect will be apparent from and elucidated with reference to the illustrations described hereinafter in which:

FIG. 1 illustrates a system including an intraoral scanning device;

FIG. 2 illustrates an exemplary intraoral scanning device;

FIG. 3A shows examples of client device certificate key;

FIG. 3B illustrates an exemplary intraoral scanning device certificate;

FIG. 4 illustrates an exemplary sequence diagram;

FIG. 5 illustrates an exemplary flowchart of a method;

FIG. 6 Schematically illustrates an exemplary architecture according to this disclosure;

FIG. 7 Schematically illustrates an exemplary client device;

FIG. 8 Schematically illustrates an exemplary client device certificate;

FIG. 9 schematically illustrates an exemplary client device certificate;

FIG. 10 schematically illustrates an exemplary signaling diagram;

FIG. 11 schematically illustrates an exemplary signaling diagram;

FIG. 12 schematically illustrates an exemplary signaling diagram;

FIG. 13 schematically illustrates a flowchart of an exemplary method; and

FIG. 14 schematically illustrates an exemplary signaling diagram.

### DETAILED DESCRIPTION

The detailed description set forth below in connection with the appended drawings is intended as a description of various configurations. The detailed description includes specific details for the purpose of providing a thorough understanding of various concepts. However, it will be apparent to those skilled in the art that these concepts may be practiced without these specific details. Several aspects of the devices, systems, mediums, programs and methods are described by various blocks, functional units, modules, components, circuits, steps, processes, algorithms, etc. (collectively referred to as “elements”). Depending upon particular application, design constraints or other reasons, these elements may be implemented using electronic hardware, computer program, or any combination thereof.

The electronic hardware may include microprocessors, microcontrollers, digital signal processors (DSPs), field programmable gate arrays (FPGAs), programmable logic devices (PLDs), gated logic, discrete hardware circuits, and other suitable hardware configured to perform the various functionality described throughout this disclosure. Computer program shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise.

A scanning for providing intraoral scan data may be performed by a dental scanning system that may include an intraoral scanning device such as the TRIOS series scanners from 3Shape A/S. The dental scanning system may include a wireless capability as provided by a wireless network unit. The intraoral scanning device may employ a scanning principle such as triangulation-based scanning, confocal scanning, focus scanning, ultrasound scanning, x-ray scanning, stereo vision, structure from motion, optical coherent tomography OCT, or any other scanning principle. In an embodiment, the intraoral scanning device is operated by projecting a pattern and translating a focus plane along an optical axis of the intraoral scanning device and capturing a plurality of 2D images at different focus plane positions such that each series of captured 2D images corresponding to each focus plane forms a stack of 2D images. The acquired 2D images are also referred to herein as raw 2D images, wherein raw in this context means that the images have not been subject to image processing. The focus plane position is preferably shifted along the optical axis of the scanning system, such that 2D images captured at a number of focus plane positions along the optical axis form said stack of 2D images (also referred to herein as a sub-scan) for a given view of the object, i.e. for a given arrangement of the scanning system relative to the object. After moving the intraoral scanning device relative to the object or imaging the object at a different view, anew stack of 2D images for that view may be captured. The focus plane position may be varied by means of at least one focus element, e.g., a moving focus lens. The intraoral scanning device is generally moved and angled during a scanning session, such that at least some sets of sub-scans overlap at least partially, in order to enable stitching in the post-processing. The result of stitching is the digital 3D representation of a surface larger than that which can be captured by a single sub-scan, i.e. which is larger than the field of view of the 3D scanning device. Stitching, also known as registration, works by identifying overlapping regions of 3D surface in various sub-scans and transforming sub-scans to a common coordinate system such that the overlapping regions match, finally yielding the digital 3D model. An Iterative Closest Point (ICP) algorithm may be used for this purpose. Another example of an intraoral scanning device is a triangulation scanner, where a time varying pattern is projected onto the dental object and a sequence of images of the different pattern configurations are acquired by one or more cameras located at an angle relative to the projector unit.

The intraoral scanning device comprises one or more light projectors configured to generate an illumination pattern to be projected on a three-dimensional dental object during a scanning session. The light projector(s) preferably comprises a light source, a mask having a spatial pattern, and one or more lenses such as collimation lenses or projection lenses. The light source may be configured to generate light of a single wavelength or a combination of wavelengths (mono- or polychromatic). The combination of wavelengths may be produced by using a light source configured to produce light (such as white light) comprising different wavelengths. Alternatively, the light projector(s) may comprise multiple light sources such as LEDs individually producing light of different wavelengths (such as red, green, and blue) that may be combined to form light comprising the different wavelengths. Thus, the light produced by the light source may be defined by a wavelength defining a specific color, or a range of different wavelengths defining a combination of colors such as white light. In an embodiment, the intraoral scanning device comprises a light source configured for exciting fluorescent material of the teeth to obtain fluorescence data from the dental object. Such a light source may be configured to produce a narrow range of wavelengths. In another embodiment, the light from the light source is infrared (IR) light, which is capable of penetrating dental tissue. The light projector(s) may be DLP projectors using a micro mirror array for generating a time varying pattern, or a diffractive optical element (DOF), or back-lit mask projectors, wherein the light source is placed behind a mask having a spatial pattern, whereby the light projected on the surface of the dental object is patterned. The back-lit mask projector may comprise a collimation lens for collimating the light from the light source, said collimation lens being placed between the light source and the mask. The mask may have a checkerboard pattern, such that the generated illumination pattern is a checkerboard pattern. Alternatively, the mask may feature other patterns such as lines or dots, etc.

The intraoral scanning device preferably further comprises optical components for directing the light from the light source to the surface of the dental object. The specific arrangement of the optical components depends on whether the intraoral scanning device is a focus scanning apparatus, a scanning device using triangulation, or any other type of scanning device. A focus scanning apparatus is further described in EP 2 442 720 B1 by the same applicant, which is incorporated herein in its entirety.

The light reflected from the dental object in response to the Illumination of the dental object is directed, using optical components of the intraoral scanning device, towards the image sensor(s). The image sensor(s) are configured to generate a plurality of images based on the incoming light received from the illuminated dental object. The image sensor may be a high-speed image sensor such as an image sensor configured for acquiring images with exposures of less than 1/1000 second or frame rates in excess of 250 frames pr. Second (fps). As an example, the image sensor may be a rolling shutter (CCD) or global shutter sensor (CMOS). The image sensor(s) may be a monochrome sensor including a color filter array such as a Bayer filter and/or additional filters that may be configured to substantially remove one or more color components from the reflected light and retain only the other non-removed components prior to conversion of the reflected light into an electrical signal. For example, such additional filters may be used to remove a certain part of a white light spectrum, such as a blue component, and retain only red and green components from a signal generated in response to exciting fluorescent material of the teeth.

The network unit may be configured to connect the dental scanning system to a network comprising a plurality of network elements including at least one network element configured to receive the processed data. The network unit may include a wireless network unit. The wireless network unit may be configured to wirelessly connect the dental scanning system to the network comprising the plurality of network elements including the at least one network element configured to receive the processed data.

The dental scanning system preferably further comprises a processor configured to generate scan data (such as intraoral scan data) by processing the two-dimensional (2D) images acquired by the intraoral scanning device. The processor may be part of the intraoral scanning device. As an example, the processor may comprise a Field-programmable gate array (FPGA) and/or an Advanced RISC Machines (ARM) processor located on the intraoral scanning device. The scan data comprises information relating to the three-dimensional dental object. The scan data may comprise any of 2D images, 3D point clouds, depth data, texture data, intensity data, color data, and/or combinations thereof. As an example, the scan data may comprise one or more point clouds, wherein each point cloud comprises a set of 3D points describing the three-dimensional dental object. As another example, the scan data may comprise images, each image comprising image data e.g. described by image coordinates and a timestamp (x, y, t), wherein depth information can be inferred from the timestamp. The image sensor(s) of the intraoral scanning device may acquire a plurality of raw 2D images of the dental object in response to illuminating said object using the one or more light projectors. The plurality of raw 2D images may also be referred to herein as a stack of 2D images. The 2D images may subsequently be provided as input to the processor, which processes the 2D images to generate scan data. The processing of the 2D images may comprise the step of determining which part of each of the 2D images are in focus in order to deduce/generate depth information from the images. The depth information may be used to generate 3D point clouds comprising a set of 3D points in space, e.g., described by cartesian coordinates (x, y, z). The 3D point clouds may be generated by the processor or by another processing unit. Each 2D/3D point may furthermore comprise a timestamp that indicates when the 2D/3D point was recorded, i.e., from which image in the stack of 2D images the point originates. The timestamp is correlated with the z-coordinate of the 3D points, i.e., the z-coordinate may be inferred from the timestamp. Accordingly, the output of the processor is the scan data, and the scan data may comprise image data and/or depth data, e.g. described by image coordinates and a timestamp (x, y, t) or alternatively described as (x, y, z). The intraoral scanning device may be configured to transmit other types of data in addition to the scan data. Examples of data include 3D information, texture information such as infra-red (IR) images, fluorescence images, reflectance color images, x-ray images, and/or combinations thereof.

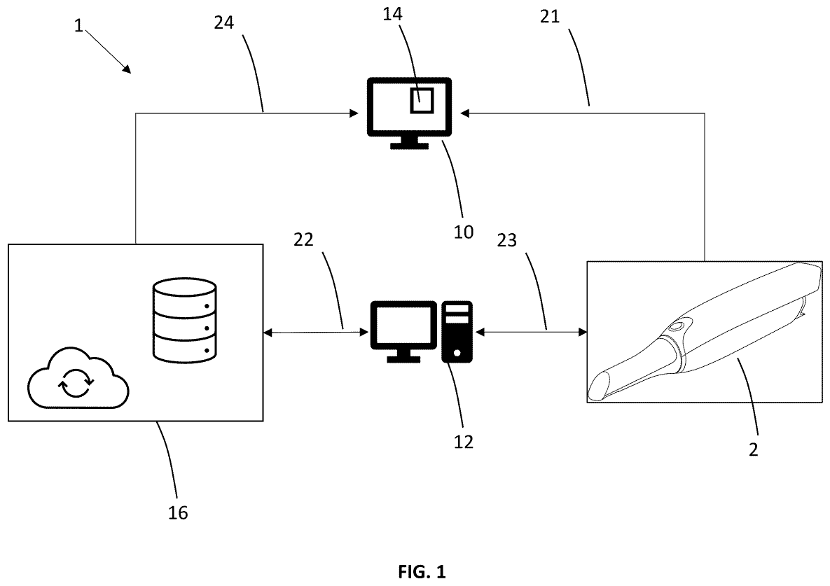

FIG. 1 illustrates exemplary devices that may be used for manufacturing, maintenance, and/or operating an intraoral scanning device 2. FIG. 1 shows an exemplary system 1 and an intraoral scanning device 2. The system 1 may comprise one or more of a manufacturing device 12, a client device 10, and a server device 16 for manufacturing, maintenance, and/or operating the intraoral scanning device 2 in connection with processing intraoral scan data of a patient and providing 2D image data and/or 3D image data.

The manufacturing device 12 may be configured to perform any steps of the method of manufacturing an intraoral scanning device. The manufacturing device 12 may be configured to generate an intraoral scanning device certificate including the intraoral scanning device identifier and at least one of the generated intraoral scanning device keys. The manufacturing device 12 may be configured to transmit the intraoral scanning device certificate to the intraoral scanning device. The manufacturing device 12 may comprise processing elements (such as a processor and a memory)

The intraoral scanning device 2 is configured to perform intraoral scan data and provide 2D image data and/or 3D image data based on processed intraoral scan data. The intraoral scanning device 2 may be configured to communicate with the manufacturing device 12 using e.g. a wireless communication link 23, such as a uni or bidirectional wireless communication link. The wireless communication link may be carried over a short-range communication system, such as WIFI, Bluetooth, or Bluetooth low energy.

The intraoral scanning device 2 may be configured to connect to the client device 10 via a wireless communication link 21, such as a bidirectional wireless communication link. The wireless communication link 21 may be carried over a short-range communication system, such as WIFI, Bluetooth, or Bluetooth low energy. The intraoral scanning device 2 may be configured to connect to the client device 10 over a network. The client device 10 may permit remote updates, such as firmware update, customization update, debug update of the intraoral scanning device where a dispenser connects to the intraoral scanning device via the client device 10 of the user. The client device 10 may comprise a computing device acting as a client, such as a customization device 14 (e.g. a handheld device, a relay, a tablet, a clinic computer, and/or USB dongle plugged in a personal computer). The processing unit/intraoral scanning device is configured to receive a linking request for a session via the interface; and to obtain a session identifier. For example, the interface of the intraoral scanning device 2 is configured to receive the linking request from the client device 10 via communication link 21. For example the intraoral scanning device 2 receives the linking request from the client device 10 for establishing a communication session. The processing unit of the intraoral scanning device is configured to transmit via the interface a linking response comprising an intraoral scanning device identifier and the session identifier. The processing unit of the intraoral scanning device is configured to receive, via the interface, an authentication message comprising an authentication key identifier and client device data, e.g. from the client device 10 via communication link 21.

The client device 10 may be configured to communicate with the server device 16 via a communication link 24, such as a bidirectional communication link. The communication link 24 may be a wired link and/or wireless communication link. The communication link 24 may comprise a network, such as the Internet.

The client device 10 may be configured to communicate with the server device 16 for maintenance, and update purposes. The server device 16 may comprise a computing device configured to act as a server, i.e. to serve requests from the client device 10 and/or from the intraoral scanning device 2. The server device 16 may be controlled by the intraoral scanning device manufacturer. The server device 16 may be configured to communicate with the manufacturing device 12 via a communication link 22 for manufacturing maintenance, and/or operational purposes. The server device 16 and the manufacturing device 12 may be co-located and/or form one entity for manufacturing maintenance, and/or operational purposes of the intraoral scanning device 2.

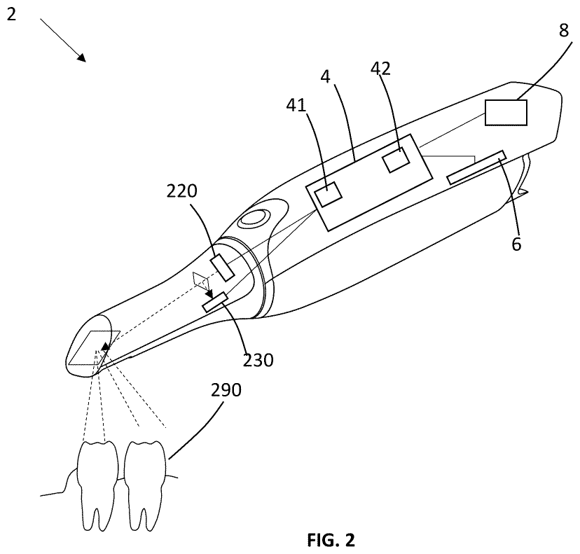

FIG. 2 illustrates an exemplary intraoral scanning device 2. The intraoral scanning device 2 comprises a processing unit 4, a memory unit 6 and a wireless interface 8. The intraoral scanning device 2 comprises a processing unit 4 configured to process intraoral scan data of a patient and provide 2D image data and/or 3D image data. The wireless interface 8 comprises a wireless transceiver, e.g. configured for wireless communication at frequencies in the range from 2.4 to 2.5 GHz, 2.4 GHz to 5 GHz, about 2.45 GHz or about 5 GHz. The wireless interface 8 is optionally configured for wireless communication with a manufacturing device 12. The processing unit 4 may be configured to provide 2d image data and/or 3D image data based on intraoral scan data according to data received during manufacture and/or updates, such as customization updates, firmware updates or debug updates. The intraoral scanning device optionally comprises a light projector 220 and an image sensor 230. The light projector includes at least one or more light emitting diodes and/or one or more infrared light sources for emitting light pattern to a three-dimensional dental object 290 of a patient or of a wax model 290 which is a replicate of the patient's dental. The image sensor 230 receives the reflective light from the dental object 290, and the image sensor 230 converts the reflected light into intraoral scan data. The processing unit 4 is then configured to process the intraoral scan data to 2D image data and/or 3D image data. The image data is then forwarded to the wireless interface 8 which transmits the data to an external device.

The processing unit 4 is configured to receive a linking request for a session via the wireless interface 8; and to obtain a session identifier. Hence, the processing unit 4 comprises e.g. an obtain unit 41 configured to obtain a session identifier. Examples of an obtain unit 41 include a random or pseudo-random number generator. The wireless interface is configured to receive the linking request for a session from a client device 10. The processing unit 4 is configured to obtain a session identifier, such as by generating a random or pseudo-random number. The processing unit 4 is configured to store the session identifier in the memory unit 6. The memory unit 6 may be configured to store the session identifier at a memory address of the memory unit 6, and/or in memory cells of the memory unit 6, such as in designated memory cells and/or at designated addresses. The linking request may comprise an authentication key identifier and/or an authentication type identifier, in order to permit the intraoral scanning device 2 to perform authentication at this early stage the linking request and the client device 10 sending the linking request. This may provide a level of access control.

The processing unit 4 is configured to transmit via the wireless interface 8 a linking response comprising an intraoral scanning device identifier and the session identifier. The processing unit 4 may be configured to generate a linking response by including the session identifier and the intraoral scanning device identifier in the linking response. The intraoral scanning device identifier may refer to a unique identifier of the intraoral scanning device 2, such as a serial number, a MAC address, and/or hardware identifier of the intraoral scanning device 2. The wireless interface 8 is configured to transmit the linking response to e.g. the client device 10.

The processing unit 4 is configured to receive, via the wireless interface 8, an authentication message comprising an authentication key identifier and client device data. For example, the wireless interface 8 may be configured to receive the authentication message from the client device 10. For example, the intraoral scanning device 2 receives the authentication message from the client device 10 in order to establish a communication session. The client device data may comprise a client device certificate (encrypted or unencrypted), customization data, intraoral scanning device operating parameters, and/or firmware data. For example, the authentication message may comprise an authentication key identifier in plain text. The authentication key identifier may be indicative of an intraoral scanning device key. The processing unit 4 that processes the authentication key identifier is configured to e.g. verify the authentication key identifier by comparing it to the intraoral scanning device key identifier stored e.g. in the memory unit 6 and determining the authentication key identifier as acceptable if the authentication key identifier is for example equal or higher than the intraoral scanning device key identifier stored.

The processing unit 4 is configured to select an intraoral scanning device key from a plurality of intraoral scanning device keys in the memory unit 6 based on the authentication key identifier. Hence, the processing unit 4 comprises e.g. a select unit 42 configured to select an intraoral scanning device key based on the authentication key identifier. When the authentication key identifier is acceptable by the intraoral scanning device 2 based on an intraoral scanning device key identifier held by the intraoral scanning device 2, the processing unit 4 is configured to select an intraoral scanning device key that the authentication key identifier indicates and to use the selected intraoral scanning device key as keying material used to secure the session. Optionally, the processing unit 4 may be configured to select an intraoral scanning device key from a plurality of intraoral scanning device keys in the memory unit 6 based on the authentication key identifier and an authentication type identifier. The authentication type identifier may be included in the authentication message and received in plaintext by the intraoral scanning device 2, and/or as client device type identifier in the client certificate (encrypted or decrypted). For example, the processing unit 4 may be configured to select an intraoral scanning device key that the authentication key identifier and the authentication type identifier indicate.

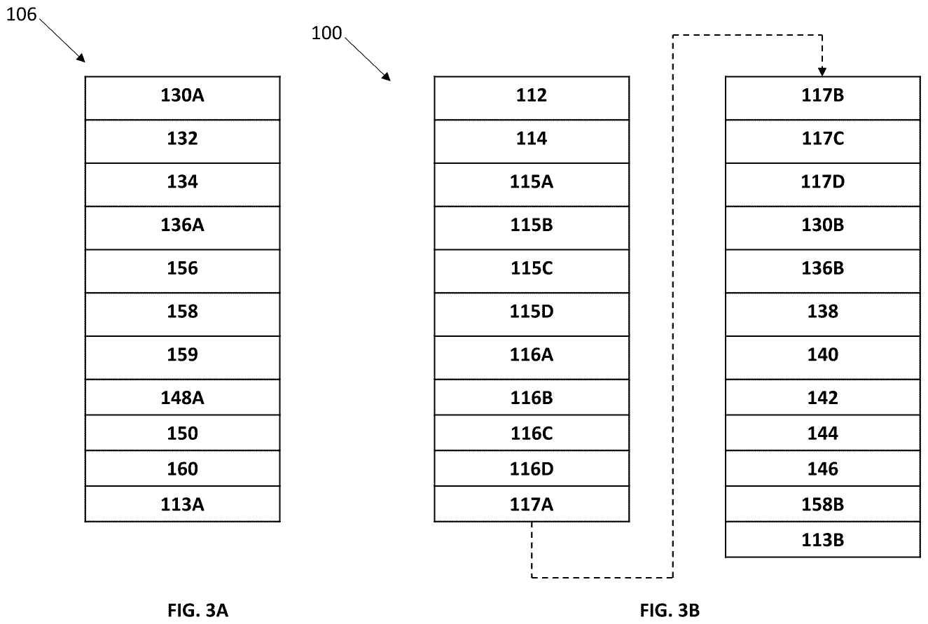

The processing unit 4 is configured to verify the client device data based on the selected intraoral scanning device key; and to terminate the session if verification fails. For example, the processing unit 4 is configured to verify the client device data based on the selected intraoral scanning device key by verifying the integrity of the client device data based on the selected intraoral scanning device key, such as verifying a MAC and/or a digital signature of the client device data. The processing unit 4 is configured to verify the client device data based on the selected intraoral scanning device key by decrypting the client device data using the selected intraoral scanning device key (as keying material to derive a decryption key or as a decryption key), when the client device data is received encrypted, and by verifying the content of the decrypted client device data. The processing unit 4 may be configured to verify the client device data based on the selected intraoral scanning device key by comparing the decrypted client device data with data stored in the memory unit 6. The client device data may comprise a client device certificate (such as an encrypted client device certificate), an authentication key identifier, and/or an authentication type identifier. The client device 10 may be assigned a client device certificate. The client device certificate refers to a certificate generated and assigned to the client device by e.g. a manufacturing device 12. Examples of client device certificates are illustrated in FIG. 3A. The processing unit 4 may be configured to generate a certificate key based on the selected intraoral scanning device key and/or the session identifier. To verify the client device data may comprise to decrypt the encrypted client device certificate with the certificate key to obtain a decrypted version of the encrypted client device certificate.

The processing unit 4 may be configured to verify the client device data by determining if the authentication key identifier matches a client device key identifier of the (decrypted) client device certificate, and verification fails if no match is determined.

In one or more exemplary intraoral scanning devices, the processing unit 4 is configured to verify the client device data by determining if a client device type identifier of the client device certificate is valid and verification fails if the client device type identifier of the client device is not valid. The processing unit 4 is configured to verify the client device data by verifying a digital signature of the client device certificate included in the client device data, and verification fails if the digital signature is not verified.

The processing unit 4 may be configured to verify the client device data by determining if the signing device identifier and/or the client device identifier are valid, e.g. not black-listed.

In one or more exemplary intraoral scanning devices, the processing unit 4 is configured to generate an offline session key based on the common secret and the session identifier, and the processing unit 4 is configured to communicate with the client device using the offline session key.

In one or more exemplary intraoral scanning devices, the authentication message 421 comprises an authentication token identifier, and the processing unit 4 is configured to store the authentication token identifier in the memory unit 6 and to link the authentication token identifier with the common secret. The authentication token identifier may be indicative of enabling a token-based authentication at the intraoral scanning device 2, i.e. when the intraoral scanning device receives an authentication token identifier from an authenticated client device 10, it may enable token-based authentication in future communication with the same client device 10 by storing e.g. an indicator such as a flag in relation with the common secret and the client device. For example, the intraoral scanning device 2 receiving the authentication token identifier may be configured to indicate to the processing unit 4 to enable token-based authentication by storing and/or linking the token identifier with the common secret generated for the same client device 10, such as by storing and/or linking the token identifier with the common secret and the client device identifier of the same client device in e.g. a table.

In the intraoral scanning device 2, the processing unit 4 is configured to generate a session key based on the session identifier and the intraoral scanning device key, and the processing unit 4 is configured to receive and authenticate session data based on the session key.

In one or more exemplary intraoral scanning devices, the processing unit 4 is configured to receive an additional authentication message via the wireless interface 8. The additional authentication message comprises client device data and an authentication device identifier. The processing unit 4 may be configured to obtain a common secret based on the authentication device identifier from the memory unit 6. The processing unit 4 may be configured to generate an additional certificate key from the common secret; and to verify the client device data based on the additional certificate key.

FIG. 3A illustrates an exemplary client device certificate 106. The client device data may comprise a client device certificate 106 and/or an encrypted client device certificate 106A. The client device 10 may be assigned a client device certificate 106. The client device certificate 106 refers to a certificate generated and assigned to the client device 10 by e.g. a manufacturing device 12. The encrypted client device certificate 106A may be generated by the client device 10 using an encryption algorithm and a certificate key.

The client device certificate 106 comprises a certificate type identifier 130A. The certificate type identifier 130A may indicate a type of the certificate amongst a variety of certificate types, such as an intraoral scanning device family certificate type, an intraoral scanning device certificate type, a firmware certificate type, a research and development certificate type, client device certificate type. The certificate type identifier 130A may be used by the intraoral scanning device 2 to identify what type of certificate it receives, stores, and/or retrieves and to act accordingly. The client device certificate 106 may comprise a version identifier 132 which indicates a data format version of the client device certificate 106. The intraoral scanning device 2 may be configured to use the certificate type identifier 130A and/or the version identifier 132 to determine what type of data the certificate comprises, and/or what type of data is comprised in a field of the certificate. For example, the intraoral scanning device 2 determines based on the certificate type identifier 130A and/or version identifier 132 what field of the certificate 106 comprises a digital signature 113A, and/or which public key is needed to verify the digital signature 113A. It may be envisaged that there is a one-to-one mapping between the certificate type identifier 130A and the public-private key pair. It may be envisaged that the intraoral scanning device 2 obtains the corresponding public key, such as retrieves the corresponding public key from the memory unit 6, a remote data storage, and/or receives the corresponding public key from the client device 10 and/or a server device 16. The client device certificate 106 may comprise a signing device identifier 136A. The signing device identifier 136A refers to a unique identifier identifying the device (such as a client device 10, a server device 16, an integrated circuit card, a smart card, and/or a hardware security module thereof) that has signed the client device certificate 106, e.g. during manufacture of the client device. The signing device identifier 136A may for example comprise a medium access control, MAC, address of the signing device, and/or a serial number of the signing device. The signing device identifier 136A allows for example the intraoral scanning device 2 to determine whether the signing device is e.g. black listed or not, and thus to reject certificates signed by a signing device that is black listed. The client device certificate 106 may comprise one or more hardware identifiers, such as a first hardware identifier 148A, and a second hardware identifier 150. The hardware identifiers, 148A, 150 may identify a piece of hardware comprised in the client device 10, such as a radio chip comprised in the client device 10, and/or a digital signal processor of the client device 10. The hardware identifier (s) may be stored in a register of the piece of hardware comprised in the intraoral scanning device during manufacturing of the piece of hardware. The hardware identifier(s) may comprise a serial number, a medium access control, MAC, address, a chip identifier, or any combination thereof In one or more exemplary client device certificates, the client device certificate 106 comprises a client device type identifier 156. A client device type identifier 156 may be indicative of a type which the client device belongs to. The client device certificate 106 may comprise a client device identifier 158. The client device certificate may comprise a client device key identifier 159. A client device key identifier 159 may be indicative of the client device key used as keying material for securing a communication with an external party.

In one or more exemplary client device certificates, the client device certificate 106 comprises a Bluetooth address or an IP address (when wireless communication is based on WIFI) 160 of the client device.

The client device certificate 106 comprises a digital signature 113A. The digital signature 113A enables a proof or verification of authenticity of the client device certificate, such as verification of the signer legitimacy. The digital signature 113A is optionally generated by the manufacturing device 12 using a client device customization private key. The intraoral scanning device 2 may be configured to verify the digital signature 113A when receiving the client device certificate comprising the digital signature 113A (i.e. receiving the authentication message comprising the encrypted client device certificate, and obtaining a decrypted version of the client device certificate 106B). The digital signature 113A is verifiable by the intraoral scanning device 2 using a corresponding client device customization public key, which is e.g. stored in the memory unit 6. If the digital signature 113A is not successfully verified using the alleged public key, the intraoral scanning device 2 may disregard the client device certificate 106 (and authentication message) and/or abort normal operation/the session. This may provide the advantage that the intraoral scanning device 2 rejects a client device certificate 106 (and authentication message) that is tampered or received from unauthenticated parties. The communication with the intraoral scanning device 2 may thus be robust against impersonation, modification and masquerading attacks.

FIG. 3B illustrates an exemplary intraoral scanning device certificate 100. The intraoral scanning device certificate 100 comprises an intraoral scanning device identifier 112, at least one intraoral scanning device key identifier including a first intraoral scanning device key identifier 114 indicative of an intraoral scanning device key and one or a plurality of intraoral scanning device keys. The intraoral scanning device identifier 112 may refer to a unique or a pseudo-unique identifier. The first intraoral scanning device key identifier 114 is indicative of the first intraoral scanning device key(s) of the intraoral scanning device certificate. For example, the first intraoral scanning device key identifier 114 may be indicative of or point to an intraoral scanning device key of a first set 115 of intraoral scanning device keys (115A, 115B, 115C, 115D) of the intraoral scanning device certificate, e.g. the first primary intraoral scanning device key 115A.

The intraoral scanning device certificate 100 optionally comprises at least four sets of intraoral scanning device keys enabling secure and distinct communication with at least four different client devices/client device types.

The intraoral scanning device certificate 100 comprises a first set 115 of intraoral scanning device keys including a first primary intraoral scanning device key 115A. The at least one intraoral scanning device key identifier comprises a first intraoral scanning device key identifier 114 indicative of an intraoral scanning device key of the first set 115 of intraoral scanning device keys 115A, 115B, 115C, 115D. The first set 115 of intraoral scanning device keys comprises for example first primary key 115A, first secondary key 115B, first tertiary key 115C, and first quaternary key 115D dedicated to securing communication to and from a first client device or a first client device type. For example, the first set 115 of intraoral scanning devices key may be a set of intraoral scanning device keys 115A, 115B, 115C, 115D for securing communication of intraoral scanning device data with the first client device.

The plurality of intraoral scanning device keys may comprise a second set 117 of intraoral scanning device keys including a second primary intraoral scanning device key 117A, a second secondary intraoral scanning device key 117B, a second tertiary intraoral scanning device key 117C, and/or a second quaternary intraoral scanning device key 117D. The at least one intraoral scanning device key identifier comprises a second intraoral scanning device key identifier 116 indicative of an intraoral scanning device key of the second set 117 of intraoral scanning device keys 117A, 117B, 117C, 117D. The intraoral scanning device is configured to communicate with one or more client devices, such as a first client device and/or a second client device. For each client device or client device type that the intraoral scanning device is configured to communicate with, the intraoral scanning device certificate optionally comprises a set of intraoral scanning device keys configured to enable secure communication with a specific client device or client device type, and an intraoral scanning device key identifier indicating which intraoral scanning device keys that are part of the intraoral scanning device certificate. The intraoral scanning device certificate may comprise a third set 119 of intraoral scanning device keys including a third primary intraoral scanning device key 119A, a third secondary intraoral scanning device key 119B, a third tertiary intraoral scanning device key 119C, and/or a third quaternary intraoral scanning device key 119D. The at least one intraoral scanning device key identifier comprises a third intraoral scanning device key identifier 118 indicative of an intraoral scanning device key of the third set 119 of intraoral scanning device keys. The intraoral scanning device certificate 100 may comprise a fourth set of intraoral scanning device keys including a fourth primary intraoral scanning device key (not shown). The at least one intraoral scanning device key identifier comprises a fourth intraoral scanning device key identifier indicative of an intraoral scanning device key of the fourth set of intraoral scanning device keys. The intraoral scanning device 2 may be configured to select a set of intraoral scanning device keys based on the client device or the client device type connected to the intraoral scanning device and to select an intraoral scanning device key from the set of intraoral scanning device keys selected based on the intraoral scanning device key identifier associated with the selected set of intraoral scanning devices.

The intraoral scanning device certificate 100 optionally comprises a certificate type identifier 130B. The certificate type identifier 130B indicates that the intraoral scanning device certificate 100 is an intraoral scanning device certificate, e.g. selected amongst a variety of certificate types, such as an intraoral scanning device family certificate type, an intraoral scanning device certificate type, a firmware certificate type, a research and development certificate type, and a client device certificate type. The certificate type identifier 130B may be used to enable the intraoral scanning device 2 to identify what type of certificate it receives, stores, authenticates and/or retrieves. The intraoral scanning device certificate 100 may comprise a version identifier which indicates a data format version of the intraoral scanning device certificate. The intraoral scanning device 2 may use the certificate type identifier 130B and/or the version identifier to determine what type of data the intraoral scanning device certificate 100 comprises, what type of data is comprised in a field of the intraoral scanning device certificate 100. For example, the intraoral scanning device 2 may determine based on the certificate type identifier 130B and/or version identifier what field of the certificate comprises a digital signature 113B, and which public key is needed to verify the digital signature 113B. It may be envisaged that there is a one-to-one mapping between the certificate type identifier 130B and the public-private key pair used for generating the digital signature 113B. The intraoral scanning device certificate 100 may comprise a length identifier that indicates the length of the intraoral scanning device certificate 100, e.g. in bits, bytes.

The intraoral scanning device certificate 100 optionally comprises a signing device identifier 136B. The signing device identifier 136B refers to a unique identifier identifying the device (such as a manufacturing device 12, e.g. an integrated circuit card, a smart card, a hardware security module comprised in a manufacturing device 12) that has signed the intraoral scanning device certificate 100. The signing device identifier 136B may for example comprise a medium access control, MAC, address of the signing device, a serial number. The signing device identifier 136B allows for example the intraoral scanning device 2 to determine whether the signing device is e.g. black-listed or not, and thus to reject intraoral scanning device certificates 100 signed by a signing device that is black-listed.

The intraoral scanning device certificate 100 optionally comprises one or more hardware identifiers including a first hardware identifier 148B and/or a second hardware identifier (not shown). The first hardware identifier 148B may identify a piece of hardware comprised in the intraoral scanning device 2, such as a processing unit 4, a radio chip comprised in the intraoral scanning device 2, a digital signal processor of the intraoral scanning device 2. The first hardware identifier 148B may also be stored in a register of the piece of hardware comprised in the intraoral scanning device 2 during manufacturing of the piece of hardware. The first hardware identifier 148B may comprise a serial number, a medium access control, MAC, address, a chip identifier, or any combination thereof. The intraoral scanning device certificate 100 may comprise a first hardware identifier 148B, a second hardware identifier and/or a third hardware identifier. For example, the first hardware identifier 148B may provide a first intraoral scanning device specific value present in a register of a hardware module (e.g. the processing unit or the radio chip) of the intraoral scanning device 2 while the second hardware identifier may provide a second intraoral scanning device specific value present in a register of a hardware module of the intraoral scanning device 2, and a third hardware identifier may provide a third hardware module identifier (e.g. a processing unit identifier, a DSP identifier). The intraoral scanning device 2, upon receiving the intraoral scanning device certificate 100 comprising the first hardware identifier 148B, may then verify the intraoral scanning device certificate 100 by comparing its stored hardware identifier and the first hardware identifier 148B comprised in the intraoral scanning device certificate 100 received. This way, the intraoral scanning device 2 may determine if the received intraoral scanning device certificate is intended for the intraoral scanning device 2 and reject the received intraoral scanning device certificate if the stored and received hardware identifiers do not match.

The intraoral scanning device certificate 100 optionally comprises a client device type authorization identifier 144. A client device type may comprise a model, category or type of client devices, such as a tablet product model, category or type, a USB dongle product model, category or type. The client device type authorization identifier 144 is an identifier of an authorized client device type, such as an identifier of the client device types that the intraoral scanning device 2 may authorize for communication, such as for customizing, maintenance and/or operation. The client device type authorization identifier 144 is for example a bit-field indicating the type of client device the intraoral scanning device 2 should allow for customization.

The intraoral scanning device certificate 100 optionally comprises a token parameter 146. The token parameter 146 indicates whether a token-based authentication is to be enabled or not. For example, if the token parameter 146 is set to 0, token-based authentication of client devices is not to be enabled by the intraoral scanning device 2 and the intraoral scanning device 2 is to use for example a combination of client device type identifier and/or a client device identifier (such as a serial number) to perform an authentication of the client device 10. If for example the token parameter 146 is set to 1, token-based authentication of client devices is to be enabled by the intraoral scanning device 2, i.e. the intraoral scanning device 2 authenticates the client device 10 (such as a based on a token received from the client device 10). The intraoral scanning device 2 may also derive a session specific token based on the received token parameter 146 which is used to e.g. accept the connection to the client device 10 without user intervention.

The intraoral scanning device certificate 100 comprises one or more of a hardware platform identifier 138, a software platform identifier 140, and/or a certificate timestamp 142. The hardware platform identifier 138 may identify a hardware platform, such as an operational intraoral scanning device hardware platform, i.e. a hardware platform on which the intraoral scanning device certificate may be used. The software platform identifier 140 may identify a family of software platforms on which the intraoral scanning device certificate is configured to operate. The certificate timestamp 142 refers to a timestamp of production or manufacture of the intraoral scanning device certificate 100, such as a timestamp of the manufacturing device 12 indicating a time instant when the intraoral scanning device certificate 100 has been generated. The certificate timestamp 142 may be in form of e.g.: hour, min, date, month, year.

The intraoral scanning device certificate comprises a digital signature 113B and/or a MAC. The digital signature 113B enables a proof or verification of authenticity of the intraoral scanning device certificate 100, such as verification of the signer legitimacy (e.g. whether the signer is a legitimate manufacturing device). The digital signature 113B is generated by the manufacturing device 12 using a device family private key during manufacturing of the intraoral scanning device. The intraoral scanning device 2 or the processing unit 4 may then verify the digital signature 113B, e.g. when receiving the intraoral scanning device certificate 100 comprising the digital signature 113B. The digital signature 113B is verifiable by the intraoral scanning device 2 using a corresponding device family public key. If the digital signature 113B is not successfully verified using the alleged public key, the intraoral scanning device may disregard the intraoral scanning device certificate 100 and/or abort normal operation.

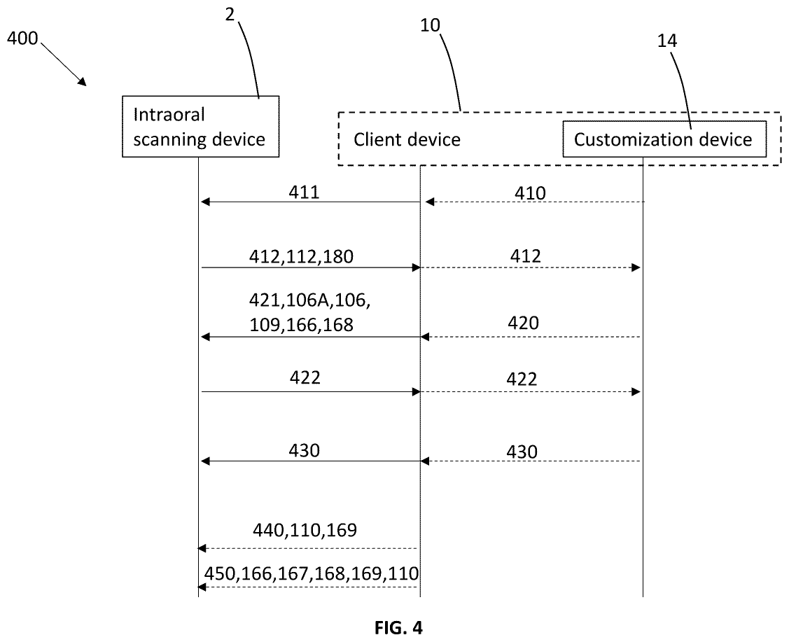

FIG. 4 illustrates an exemplary sequence diagram 400 involving an intraoral scanning device 2, and a client device 10. The client device 10 may comprise a customization device 14. The intraoral scanning device 2 receives via the wireless interface 8 a linking request or message 411 for session from the client device 10. When the client device 10 comprises a customization device 14, the customization device 14 may generate a linking request 410, which is transmitted by the client device 10 as linking request 411. The intraoral scanning device 2 obtains a session identifier 180, such as generates a session identifier 180. The intraoral scanning device 2 generates a linking response 412 comprising an intraoral scanning device identifier 112 and/or a session identifier 180 and transmits the linking response 412 to the client device 10. When the client device 10 comprises a customization device 14, the customization device 10 may receive the linking response 412 via the client device 10. The client device 10 generates an authentication message 421 and transmits the authentication message 421 to the intraoral scanning device 2. The intraoral scanning device 2 receives the authentication message 421 from the client device 10. The authentication message 421 comprises an authentication key identifier 166, optional authentication type identifier 168, and client device data 109. The client device data 109 comprises an encrypted client device certificate 106A or client device certificate 106. Any of client device type identifier 156, client device identifier 158, and a user identifier may be comprised in the encrypted client device certificate 106A. Any of a client device identifier, a client device type identifier 156 and/or a user identifier may be comprised in the authentication message 421 in plain text, or as part of (plain) client device certificate 106. When the client device 10 comprises a customization device 14, the customization device 14 may generate an authentication message 420, which is transmitted by the client device 10 as authentication message 421.

Optionally, the intraoral scanning device 2 is configured to authenticate the authentication message 421 by verifying the content, origin and/or integrity of the authentication message 421. For example, the intraoral scanning device 2 verifies whether the received value of authentication key identifier 166 received is higher or equal to an intraoral scanning device key identifier comprised in the intraoral scanning device certificate 100 (and/or the latest value the intraoral scanning device 2 has stored as intraoral scanning device key identifier in e.g. a flash memory). If the intraoral scanning device 2 determines that the received value of authentication key identifier 166 received is higher or equal to an intraoral scanning device key identifier, the authentication continues, else the session is terminated immediately (with proper error code). This prevents the intraoral scanning device 2 to communicate with an expired/revoked client device.

For example, the intraoral scanning device 2 determines the type of client device based on authentication type identifier. The intraoral scanning device 2 selects an intraoral scanning device key from a plurality of intraoral scanning device keys based on the authentication type identifier 168 and/or the authentication key identifier 166. For example, the intraoral scanning device 2 identifies the intraoral scanning device key corresponding to the authentication key identifier 166 received. The intraoral scanning device 2 uses the identified intraoral scanning device key and the session identifier 180 (e.g. a 16 bytes of random number) sent in linking response 412 to the client device 10 to derive the certificate key, such as to compute a common secret from which the certificate key is derivable.

Using the certificate key, the intraoral scanning device 2 decrypts the encrypted client device certificate 106A. The intraoral scanning device 2 may then verify that the certificate type identifier 130A comprised in the decrypted client device certificate 106B corresponds to the right certificate 106B. The intraoral scanning device 2 may then verify that the authentication type identifier 168 received in plain text match the client device type identifier 156 in the decrypted certificate 106B. The intraoral scanning device 2 may then verify that the authentication key identifier 166 received in plain text match the client device key identifier 159 in the decrypted certificate 106B and may further assess if the authentication key identifier 166 and/or the client device key identifier 159 is indicative of an intraoral scanning device key identifier (such as a first intraoral scanning device key identifier 114) held by the intraoral scanning device 2. The intraoral scanning device 2 may then verify that the version identifier 132 in the decrypted certificate 106B is supported by the intraoral scanning device 2. The intraoral scanning device 2 may then verify that the authentication type identifier 168 received in plain text or the client device type identifier 156 is listed in the client device type authorization identifier 144 of the stored intraoral scanning device certificate 100. The intraoral scanning device 2 may then verify that the authentication type identifier 168 received in plain text or the client device type identifier 156 associated with the first hardware identifier 148A, 150 is not black-listed. The signing device identifier 136A is verified not to be listed on the blacklist. The intraoral scanning device 2 may then verify the digital signature 113A of the client device certificate 106B using the matching public key.

Upon successful authentication of the authentication message 421 and/or verification, the intraoral scanning device 2 may send an authentication response 422 to the client device 10 that may forward it to a customization device 14 when the client device 10 comprises the customization device 14.

The communication channel is now open and secure. The client device 10 or customization device 14 via the client device 10 may send intraoral scanning device data 430 to the intraoral scanning device 2, such as intraoral scanning device data 430 in a session secured by a session key. Intraoral scanning device data 430 comprises e.g. firmware, customization data, and/or intraoral scanning device operating parameters. Customization data may for example be data generated by a customization device 14 used by a dentist. Customization data may comprise setting data, such as power management settings, configuration of a user interface of the intraoral scanning device and/or settings of an optical unit of the intraoral scanning device. Firmware may refer to a computer program provided by the intraoral scanning device manufacturer, and to be installed on the intraoral scanning device 2 to control the intraoral scanning device 2. Firmware is for example to be installed to upgrade the operations and capabilities of the intraoral scanning device 2.

For example, when the client device 10 re-connects to the intraoral scanning device 2, the processing unit 4 may be configured to receive via the interface 8 an additional authentication message 440. The additional authentication message 440 comprises client device data 110 and an authentication device identifier 169. The processing unit 4 may be configured to verify the authentication message 440 and authenticate the client device sending the authentication message 440. The processing unit 4 may be configured to obtain, from the memory unit 6, a common secret based on the authentication device identifier 169. The memory unit 6 may have client device identifiers 158 associated with corresponding common secrets stored thereon, for authenticated client devices 10. The processing unit 4 may then be configured to retrieve the corresponding common secret based on the authentication device identifier 169. The common secret has been generated and stored earlier at e.g. an initial round of authentication of a returning client device 10. Thus, once the client device 10 authenticated, the processing unit 4 can just retrieve the corresponding common secret from the memory unit 6. This provides a faster subsequent authentication, and avoids having to regenerate the common secret for computing the additional certificate key, and thus saves the corresponding power consumption. The processing unit 4 may be configured to generate an additional certificate key from the common secret and to verify the client device data 110 based on the additional certificate key. For example, the processing unit 4 generates the additional certificate key by computing a hash value based on the common secret and a certificate value. As described above, the processing unit 4 may be configured to verify the client device data 110 based on the additional certificate key by verifying the integrity of the client device data 110, such as verifying a MAC and/or a digital signature of the client device data 110. The processing unit 4 is configured to verify the client device data 110 based on the additional certificate key by decrypting the client device data 110 using the additional certificate key (as a decryption key), when the client device data 110 is received encrypted. The processing unit 4 is configured to verify the client device data 110 by verifying the content of the client device data 110. The processing unit 4 may be configured to verify the client device data by comparing the client device data 110 with data stored in the memory unit 6. The client device data 110 may comprise a client device certificate 106, such as an encrypted client device certificate 106A.

The processing unit 4 may be configured to receive a further authentication message 450 comprising client device data 110 and an authentication token identifier 167. The processing unit 4 is configured to obtain a common secret based on the authentication token identifier 167 from the memory unit 6; to generate a token key from the common secret.

In one or more exemplary intraoral scanning devices, the processing unit 4 is configured to receive a further authentication message 450 comprising client device data 110, an authentication type identifier 168, an authentication key identifier 166 and/or an authentication session token identifier 167. The further authentication message may comprise an authentication device identifier 169. The processing unit 4 may be configured to find in the memory unit 6 the common secret linked to the client device type identifier and/or the client device identifier of the client device 10 that sends the further authentication message 450 based on locating the stored client device type identifier corresponding to the authentication type identifier 168 and/or locating the stored client device identifier corresponding to the authentication device identifier 169. The processing unit 4 may be configured to obtain a common secret based on the authentication type identifier 168; to generate a token key based on the common secret; and to generate a session token identifier based on the token key and the session identifier. The processing unit 4 may have generated in an earlier session with the client device 10 a common secret to e.g. establish a certificate key and may have stored and linked the common secret to the client device type identifier and/or the client device identifier. The processing unit 4 may then be configured to obtain the common secret based on the authentication type identifier 168 and/or the authentication client identifier 169 corresponding to the stored client device type identifier and/or client device identifier. The processing unit 4 may be configured to generate a token key by performing a hash function on the common secret and a token value (such as a pre-defined arbitrary string or a pre-defined arbitrary value). The processing unit 4 may be configured to generate a session token identifier based on the token key and the session identifier by generating a session identifier, and by performing a hash function on the token key and the session identifier. The processing unit 4 may be configured to verify the authentication session token identifier based on the session token identifier. The processing unit 4 may be configured to verify the authentication session token identifier by comparing the authentication session token identifier 167 and the generated session token identifier. For example, if the processing unit determines that the authentication session token identifier 167 matches the generated session token identifier, the verification is successful and the processing unit 4 may proceed with no user physical intervention, and continue to verify the client device data 110 provided in the further authentication message 450. The client device data 100 may comprise a client device certificate, which may be verified according to this disclosure.

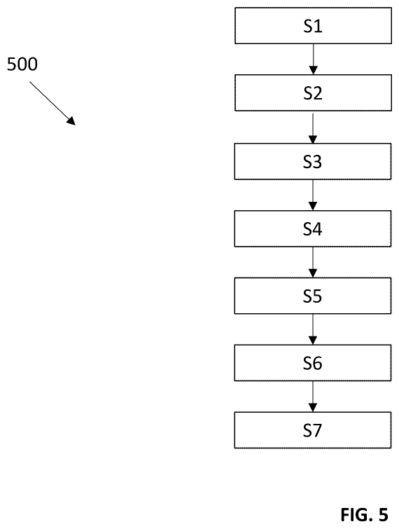

FIG. 5 illustrates a flowchart of an exemplary method 500 of operating an intraoral scanning device 2. The intraoral scanning device 2 comprises a processing unit 4 configured to process intraoral scan data and provide image data, a memory unit 6, and a wireless interface 8. The method 500 comprises receiving S1 a linking request for a session via the wireless interface 8. The linking request may comprise an authentication key identifier 166 and/or an authentication type identifier 168, in order to allow the intraoral scanning device 2 to perform authentication at this early stage the linking request and the client device sending the linking request. This may provide a level of access control.

The method 500 comprises obtaining S2 a session identifier 180, e.g. with the intraoral scanning device. Obtaining S2 a session identifier 180 may comprise generating a session identifier 180, such as by generating a random or pseudo-random number. For example, the processing unit 4 generates a random or pseudo-random number of a predetermined length, e.g. 16 bits, 32 bits, 64 bits etc., to be used as a session identifier 180. Obtaining S2 a session identifier 180 may comprise retrieving a session identifier 180 from the memory unit. The method 500 may comprise storing the session identifier 180 in the memory unit 6. For example, storing the session identifier 180 in the memory unit 6 comprises storing the session identifier 180 at a memory address of the memory unit 6, and/or in memory cells of the memory unit 6, such as in designated memory cells and/or at designated addresses.

The method 500 comprises transmitting S3 via the interface 8 a linking response comprising an intraoral scanning device identifier 112 and the session identifier 180. Transmitting S3 the linking response may comprise generating the linking response by including the session identifier 180 and the intraoral scanning device identifier 112 and transmitting the thus generated linking response to e.g. the client device 10.

The method 500 comprises receiving S4, via the interface 8, an authentication message. The authentication message comprises an authentication key identifier 166 and client device data 109. The method 500 may comprise receiving, via wireless the interface 8, an authentication message 421 from a client device 10. For example, the intraoral scanning device 2 receives the authentication message 421 from the client device 10 in order to establish a communication session. The client device data 109 may comprise a client device certificate, such as unencrypted client device certificate 106 or encrypted client device certificate 106A, customization data, intraoral scanning device operating parameters, and/or firmware data.

The authentication key identifier 166 may be an identifier that may be used to verify if the client device 10 has used a client device key acceptable by the intraoral scanning device 2.

The method 500 comprises selecting S5 an intraoral scanning device key from a plurality of intraoral scanning device keys (e.g. within or amongst a first set of intraoral scanning device keys 115, second set of intraoral scanning device keys 116 etc.) in the memory unit 6 based on the authentication key identifier 166. When the authentication key identifier 166 matches the intraoral scanning device key identifier, such as a first intraoral scanning device key identifier 114 held by the intraoral scanning device 2, the processing unit 4 may be configured to use the authentication key identifier 166 as a key identifier indicating which intraoral scanning device key is to be used as keying material in the session. Selecting S5 an intraoral scanning device key from a plurality of intraoral scanning device keys in the memory unit may be based on the authentication key identifier 166 and/or an authentication type identifier 168. The authentication type identifier 168 may be received in plaintext by the intraoral scanning device 2 as part of the authentication message 421, and/or as client device type identifier 156 in the client device certificate 106 (encrypted or decrypted). For example, selecting S5 comprises selecting an intraoral scanning device key that the authentication key identifier 166 and the authentication type identifier 168 indicate.

The method 500 comprises verifying S6 the client device data 109 based on the selected intraoral scanning device key and terminating S7 the session if verification fails. Verifying S6 the client device data 109 based on the selected intraoral scanning device key may comprise verifying the integrity of the client device data 109 based on the selected intraoral scanning device key, such as verifying a MAC and/or a digital signature comprised in the client device data 109. Verifying S6 the client device data 109 based on the selected intraoral scanning device key may comprise decrypting the client device data 109 using the selected intraoral scanning device key (as keying material to derive a decryption key or as a decryption key), when the client device data 109 is received encrypted. Verifying S6 the client device data 109 based on the selected intraoral scanning device key may comprise verifying the client device data 109 by comparing the received client device data 109 with data stored in the memory unit. For example, verification fails and the session is terminated if integrity of the client device data 109 is detected as corrupted by e.g. verifying a MAC or a digital signature, if decryption fails, and/or if comparison of the received client device data 109 (when decrypted if the client device data comprises encrypted data, such as encrypted client device certificate) with data stored in the memory unit, e.g. intraoral scanning device certificate, shows a mismatch or is indicative of corruption.

The authentication message optionally comprises an authentication type identifier 168. An authentication type identifier may be indicative of a client device type identifier 156 and/or a certificate type identifier 130, e.g. included in the client device data (encrypted). Selecting S5 an intraoral scanning device key from a plurality of intraoral scanning device keys may be based on the authentication type identifier 168. Selecting S5 the intraoral scanning device key may be based on the authentication type identifier 168 and/or the authentication key identifier 166 provided in the authentication message 421.

The client device data 109 may comprise a client device certificate 106 or an encrypted client device certificate 106A, an authentication key identifier 166, and/or an authentication type identifier 168. The method 500 may comprise generating S8 a certificate key based on the selected intraoral scanning device key and/or the session identifier 180; and verifying S6 the client device data 109 may comprise decrypting the encrypted client device certificate 106A with the certificate key to obtain a decrypted version 106B of the encrypted client device certificate 106A. Decrypting the encrypted client device certificate 106A with the certificate key may comprise decrypting the encrypted client device certificate 106A using a certificate key, a common secret and/or an intraoral scanning device key, such as generating a certificate key based on a common secret, and processing the encrypted client device certificate 106A using a decryption function and a certificate key. The certificate key may be based on a common secret and/or a certificate value. Generating a certificate key may comprise obtaining or generating the common secret based on the selected intraoral scanning device key. For example, generating the common secret based on the intraoral scanning device key comprises retrieving the intraoral scanning device certificate 100 from the memory unit 6, the intraoral scanning device certificate 100 comprising the selected intraoral scanning device key, and/or retrieving the selected intraoral scanning device key from the memory unit 6. The method may comprise generating the common secret based on a session identifier 180 and/or the selected intraoral scanning device key (e.g. the first primary intraoral scanning device key 115A). For example, the common secret CS is generated based on a selected intraoral scanning device key and a session identifier 180, e.g. as follows:

$$CS = hash ( H D\_KEY , S\_ID ) ,$$

where hash is a hash function, HD_KEY is the selected intraoral scanning device key and S_ID is a session identifier 180. The session identifier 180 may comprise a random or pseudo random number of a defined length. The common secret may be used as a certificate key in one or more exemplary methods. The method 500 may comprise storing the common secret in the memory unit 6, so as to e.g. retrieve the common secret from the memory unit 6 when needed.

Generating a certificate key may comprise performing a hash function on the common secret and/or a certificate value. Generating the certificate key may be performed e.g. as follows:

$$C\_KEY = hash ( CS , C\_VAL ) ,$$

where hash is a hash function, CS is the common secret and C_VAL is a certificate value. The certificate value may be a predefined value or string, such as the string “certificate”.

In one or more exemplary methods, generating a certificate key comprises performing a hash function on the intraoral scanning device key (e.g. the first primary intraoral scanning device key 115A) and the session identifier 180. Stated differently, the common secret may be used as a certificate key if the client device has also used the common secret as certificate key to encrypt the client device certificate 106.

Verifying S6 the client device data 109 may comprise decrypting the encrypted client device certificate 106A using the certificate key generated by the intraoral scanning device 2 and obtaining the decrypted version 106B of the encrypted client device certificate 106A.

In one or more exemplary methods, verifying S6 the client device data 109 may comprise verifying a content of the decrypted version 106B of the encrypted client device certificate 106A, e.g. based on an intraoral scanning device certificate or at least parts thereof For example, verifying S6 (with the intraoral scanning device) the client device data 109 comprises determining if the authentication key identifier 166 matches a client device key identifier 159 of the client device certificate 106, and verification fails if no match is determined.

In one or more exemplary methods, verifying S6 the client device data 109 comprises determining if a client device type identifier 156 of the client device certificate 106 or 106B is valid and verification fails if the client device type identifier 156 of the client device certificate 106 or 106B is not valid. For example, an authentication type identifier 168 is sent in plain text in the authentication message 421, the authentication type identifier 168 sent in plain text is valid if the authentication type identifier 168 matches a corresponding client device type identifier 156 comprised in the decrypted version 106B of the client device certificate 106. For example, determining if a client device type identifier 156 of the client device certificate 106 is valid comprises determining if the client device type identifier 156 of the client device certificate 106 is comprised in a list of authorized client devices stored in the memory unit 6 and/or retrieved from remote data storage.

In one or more exemplary methods, verifying S6 the client device data 109 comprises verifying a digital signature 113A of the client device certificate 106, 106B, and verification fails if the digital signature is not verified. For example, the client device data 109 comprises a digital signature 113A included in or appended to the client device data 109 to protect integrity of the client device data 109. Verifying a digital signature 113A comprises e.g. computing a comparison result based on the digital signature 113A and a corresponding client device public key and comparing the comparison result to the received client device data 109. Verifying a digital signature 113A may comprise retrieving the corresponding client device public key from the memory unit 6 and/or from remote data storage. The digital signature 113A is verified as valid, or the verification is successful when the digital signature 113A raised to the power of the corresponding client device public key is identical to the received client device data 109.

In one or more exemplary methods, the client device certificate 106 comprises a signing device identifier 136A and/or a client device identifier 158, and verifying S6 the client device data 109 comprises determining if the signing device identifier 136A and/or the client device identifier 158 of the client device certificate 106 or 106B is valid and wherein verification fails if the client device identifier 158 and/or the signing device identifier 136A is not valid.

In one or more exemplary methods, the method 500 comprises receiving an additional authentication message 440 comprising client device data 110 and an authentication device identifier 169. The method may further comprise obtaining, from the memory unit 6, a common secret based on the authentication device identifier 169, generating an additional certificate key from the common secret, and verifying the client device data 110 based on the additional certificate key.

In one or more exemplary methods, the method 500 comprises generating an offline session key based on the common secret and the session identifier 180, and communicating with the client device 10 using the offline session key.

In one or more exemplary methods, the method 500, the authentication message 421 comprises an authentication token identifier, and the method 500 comprises storing the authentication token identifier in the memory unit 6 and linking the authentication token identifier with the common secret.

In one or more exemplary methods, the method 500 comprises receiving a further authentication message 450 comprising client device data 110, an authentication type identifier 168, an authentication key identifier 166 and/or an authentication session token identifier 167. The method 500 may comprise obtaining a common secret based on the authentication type identifier 168, generating a token key based on the common secret; and generating a session token identifier based on the token key and the session identifier. The method 500 may comprise verifying the authentication session token identifier 167 based on the session token identifier.

In one or more exemplary methods, the method 500 comprises generating a session key based on the session identifier 180 and the intraoral scanning device key, receiving and authenticating session data (e.g. intraoral scanning device data 430) based on the session key.
XXXXXXXc

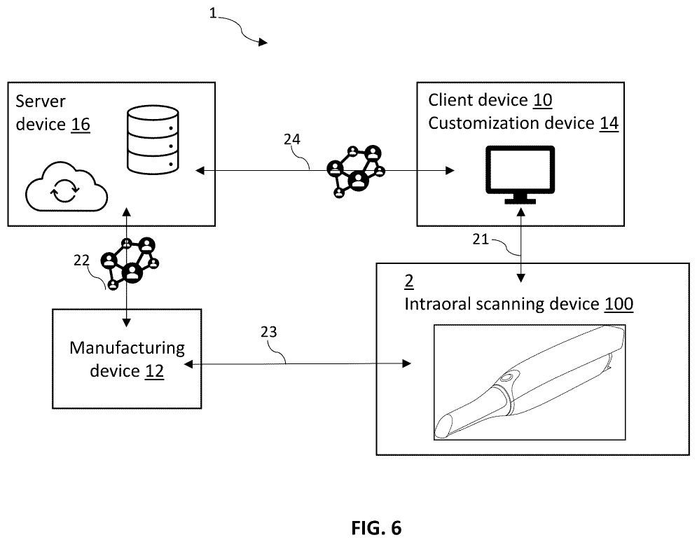

FIG. 6 schematically illustrates an exemplary architecture according to this disclosure with exemplary devices that may be used for manufacturing, maintenance, and/or operating an intraoral scanning device 2. FIG. 6 shows an exemplary system 1 and an intraoral scanning device 2. The system 1 may comprise one or more of a manufacturing device 12, a client device 10, and a server device 16 for manufacturing, maintenance, and/or operating the intraoral scanning device 2 in connection with intraoral scanning session (such as for customizing the intraoral scanning device and/or for updating an intraoral scanning device parameter).

The client device 10 may be configured to perform any acts of the method disclosed herein. The client device 10 may comprise processing elements (such as a processor and a memory) configured to perform any of the steps of the method disclosed herein. The intraoral scanning device 2 may be configured to acquire intraoral scan data from a three-dimensional dental object during a scanning session. The intraoral scanning device 2 may be configured to communicate with the client device 10 using e.g. a communication link 21. The communication link 21 may be a wireless communication link. The communication link 21 may be a single hop communication link or a multi-hop communication link. The wireless communication link may be carried over a short-range communication system, such as Bluetooth, Bluetooth low energy, IEEE 802.11. The intraoral scanning device 2 may be configured to receive an intraoral scanning device certificate from the manufacturing device 12 via communication link 23 and to store the intraoral scanning device certificate in a memory unit comprised in the intraoral scanning device 2. Alternatively or additionally, the manufacturing device 12 may store the intraoral scanning device certificate in the memory unit of the intraoral scanning device. The intraoral scanning device 2 may configured to connect to the client device 10 over a network. The client device 10 may permit remote customization of the intraoral scanning device 2, where a dispenser connects to the intraoral scanning device via the client device 10. The client device 10 may comprise a computing device acting as a client, such as a customization device 14 (e.g. a handheld device, a relay, a tablet, a personal computer, and/or USB dongle plugged in a personal computer). The client device 10 may be configured to communicate with the server device 16 via a communication link 24, such as a bidirectional communication link. The communication link 24 may be a wireless communication link. The communication link 24 may comprise a network, such as the Internet. The client device 10 may be configured to communicate with the server device 16 for maintenance, and update purposes. The server device 16 may comprise a computing device configured to act as a server, i.e. to serve requests from the client device 10 and/or from the intraoral scanning device 2. The server device 16 may be controlled by the intraoral scanning device manufacturer. The server device 16 may be configured to communicate with the manufacturing device 12 via a communication link 22 for manufacturing maintenance, and/or operational purposes. The server device 16 and the manufacturing device 12 may be co-located and/or form one entity for manufacturing maintenance, and/or operational purposes of the intraoral scanning device 2.

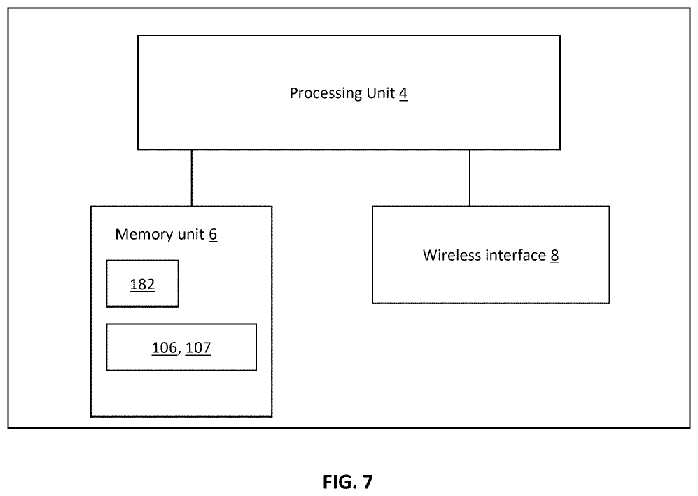

FIG. 7 schematically illustrates an exemplary client device 10. The client device 10 comprises a processing unit 4, a memory unit 6 and an wireless interface 8. The wireless interface 8 comprises a wireless transceiver, e.g. configured for wireless communication at frequencies in the range from 2.4 to 2.5 GHz. The wireless interface 8 is configured for communication, such as wired and/or wireless communication, with an intraoral scanning device 2 and/or a server device. The memory unit 6 has a client device key 182 and a client device certificate 106, 107 stored thereon. The processing unit 4 is configured to receive a connection response comprising an intraoral scanning device identifier via the wireless interface 8 and optionally to obtain a session identifier, e.g. as part of the connection response. A connection response including both the session identifier and the intraoral scanning device identifier reduces the risk of intervention from an attacker. Further, the number of connection responses from an intraoral scanning device is reduced, thereby reducing power consumption in the intraoral scanning device. The processing unit 4 is configured to generate one or more keys including a certificate key based on the intraoral scanning device identifier and/or the session identifier. The processing unit 4 is configured to generate one or more keys including a certificate key optionally based on the client device key. In the illustrated client device 10, the certificate key is generated by performing one or more hash functions. For example, the certificate key, C_KEY, may be given as:

$$C\_KEY = hash ( CS , C\_VAL ) ,$$

where hash is a hash function, CS is a common secret and C_VAL is a certificate value, e.g. a predefined value or string, such as “certificate”. In the exemplary client device 10, the common secret, CS, is based on the intraoral scanning device key and the session identifier, e.g. given as:

$$CS = hash ( IOS\_KEY , S\_ID ) ,$$

where hash is a hash function, IOS_KEY is the intraoral scanning device key and SID is the session identifier. The intraoral scanning device key, IOS_KEY, is based on the intraoral scanning device identifier and the client device key, e.g. given as:

$$IOS\_KEY = hash ( IOS\_ID , CD\_KEY ) ,$$

where hash is a hash function, IOS_ID is the intraoral scanning device identifier and CD_KEY is the client device key.

The certificate value may be a predefined value or string, such as “certificate”. Generating the certificate key may comprise performing a hash function on the common secret and/or the certificate value. For example, the certificate key, C_KEY, may be given as:

$$C\_KEY = hash ( CS , C\_VAL ) ,$$

where hash is a hash function, CS is the common secret and C_VAL is the certificate value.

The processing unit 4 is configured to obtain an authentication message based on the certificate key and the client device certificate. To obtain the authentication message comprises to generate an encrypted client device certificate by encrypting the client device certificate with the certificate key and to include the encrypted client device certificate in the authentication message. To obtain the authentication message comprises to include an authentication key identifier and/or an authentication type identifier in the authentication message. The authentication key identifier is a copy of or at least indicative of the client device key identifier. The authentication type identifier is a copy of or at least indicative of the client device type identifier. The use of authentication identifier(s), such as authentication key identifier and/or authentication type identifier in the authentication message enables an intraoral scanning device to select the correct keying material for decrypting the encrypted client device certificate and/or check whether the authentication message is generated by an outdated client device. Further, the processing unit 4 is configured to transmit the authentication message to the intraoral scanning device via the wireless interface 8.

In the exemplary processing unit 4, to generate one or more keys comprises to generate a session key based on the intraoral scanning device identifier, the session identifier and the client device key, and wherein the processing unit is optionally configured to transmit the session key to a customization device. When the client device 10 comprises the customization device, the session key is used for data communication with the intraoral scanning device.

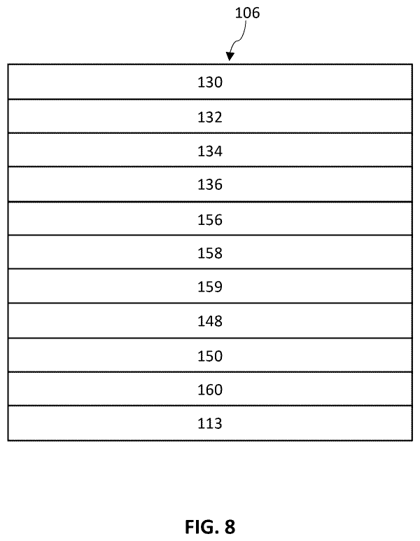

FIG. 8 schematically illustrates an exemplary client device certificate 106. The client device certificate 106 comprises a client device identifier 158 and a client device key identifier 159. The client device identifier 158 enables an intraoral scanning device to check if the client device has been black-listed. The client device key identifier 159 is indicative of the client device key (stored in the memory unit) used for generating the certificate key. The client device key identifier 159 of the client device certificate enables an intraoral scanning device to check the validity of the authentication key identifier of the authentication message.

The client device certificate 106 comprises a digital signature 113 and/or a MAC. The digital signature 113 enables a proof or verification of authenticity of the client device certificate 106, such as verification of the signer legitimacy (e.g. whether the signer is a legitimate manufacturing device). The digital signature 113 is generated during manufacture, e.g. using a device family private key during manufacturing of the client device. The client device 10 or the processing unit 4 may verify the digital signature 113 when receiving the client device certificate 100 comprising the digital signature 113. The digital signature 113 is verifiable by the client device 10 and/or an intraoral scanning device using a corresponding device family public key, e.g. selected according to the certificate type identifier. If the digital signature 113 is not successfully verified using the alleged public key, the client device 10 may abort normal operation.

The client device certificate 106 comprises a certificate type identifier 130. The certificate type identifier 130 indicates that the client device certificate 106 is a client device certificate, e.g. selected amongst a variety of certificate types, such as an intraoral scanning device family certificate type, an intraoral scanning device certificate type, a firmware certificate type, an access right certificate type, and a client device certificate type. The certificate type identifier 130 may be used to enable an intraoral scanning device 2 to identify what type of certificate it receives, stores, authenticates and/or retrieves. The client device certificate 106 may comprise a version identifier 132 which indicates a data format version of the client device certificate 106. An intraoral scanning device 2 may use the certificate type identifier 130 and/or the version identifier 132 to determine what type of data the client device certificate 106 comprises and/or what type of data is comprised in a field of the client device certificate 106. For example, an intraoral scanning device may determine based on the certificate type identifier 130 and/or version identifier 132 what field of the client device certificate comprises a digital signature 113, and which public key from a plurality of public keys is to be used to verify the digital signature 113. It may be envisaged that there is a one-to-one mapping between the certificate type identifier 130 and the public-private key pair used for generating the digital signature 113. The intraoral scanning device certificate 106 may comprise a length identifier 134 that indicates the length of the client device certificate 106.

The client device certificate 106 optionally comprises a signing device identifier 136. The signing device identifier 136 refers to a unique identifier identifying the device (such as a an integrated circuit card, a smart card, a hardware security module comprised in or connected to a manufacturing device) that has signed the client device certificate 106. The signing device identifier 136 may for example comprise a medium access control, MAC, address of the signing device and/or a serial number. The signing device identifier 136 allows for example an intraoral scanning device 2 to determine whether the signing device of the client device certificate is e.g. black-listed or not, and thus to reject client device certificates 106 signed by a signing device that is black-listed.

The client device certificate 106 optionally comprises one or more hardware identifiers including a first hardware identifier 148 and/or a second hardware identifier 150. The hardware identifiers 148, 150 may respectively identify a piece of hardware comprised in the client device 10, such as a processing unit 4 or a radio chip comprised in the wireless interface 4. The first hardware identifier 148 and/or the second hardware identifier 150 may also be stored in a register of the piece of hardware comprised in the client device 10 during manufacturing of the piece of hardware. The first hardware identifier 148 and/or the second hardware identifier 150 may comprise a serial number, a medium access control, MAC, address, a chip identifier, or any combination thereof. For example, the first hardware identifier 148 may provide a first client device specific value present in a register of a hardware module (e.g. the processing unit or the radio chip) of the client device 10 while the second hardware identifier may provide a second client device specific value present in a register of a hardware module of the client device 10.

The client device certificate 106 comprises a client device type identifier 156. The client device type identifier 156 indicates a type of the client device amongst a variety of client device types, such as a model, category or type of client devices, such as a customization type, e.g. a tablet product model, category or type for customizing the intraoral scanning device, a USB dongle product model, category or type for customizing the intraoral scanning device. Optionally, the client device certificate 106 comprises a bluetooth address 160 or at least part thereof, e.g. assigned by the manufacturer during manufacture. Addition of one or more fields and/or identifiers to the client device certificate is contemplated e.g. for a second generation client device certificate.

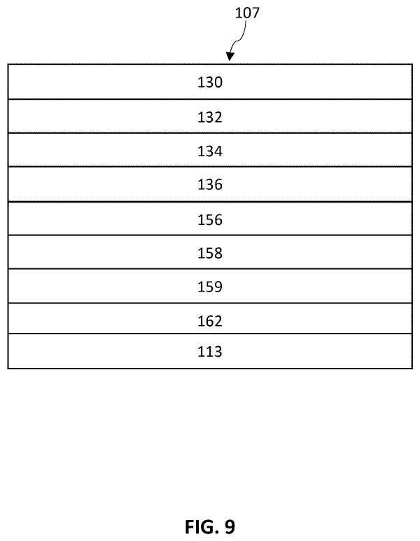

FIG. 9 schematically illustrates an exemplary client device certificate 107. The client device certificate 107 comprises certificate type identifier 130, optional version identifier 132, optional length identifier 134 and optional signing device identifier 136 as described above for client device certificate 106. The client device certificate 107 comprises client device type identifier 156, client device identifier 158, client device key identifier 159, and a user identifier 162. The user identifier 162 may be a in the form of a user name. Client device certificate 107 with a user identifier 162 may facilitate the use of a generic device, such as a tablet computer, as a client device, e.g. by implementing a user verification/key generation/certificate encryption/decryption at a remote server device, such as server device 16 controlled by intraoral scanning device manufacturer.

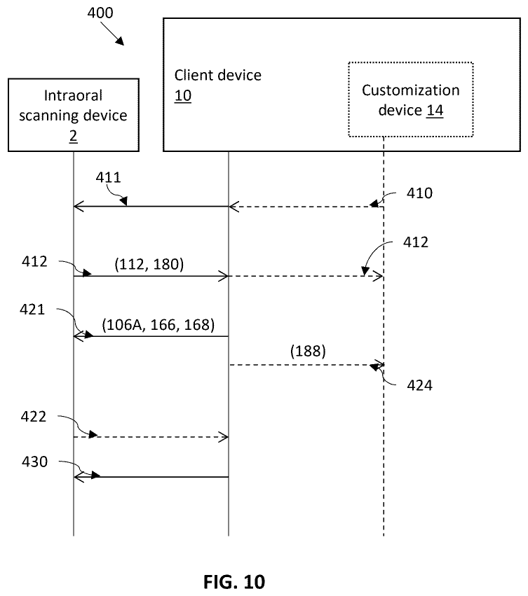

FIG. 10 schematically illustrates an exemplary signaling diagram 400 involving an intraoral scanning device 2 and a client device 10. The client device 10 may comprise a customization device 14 or be connected to a customization device 14. The client device 10 transmits a connection request or message 411 to intraoral scanning device 2. When the client device 10 comprises a customization device 14, the customization device 14 may generate a connection request 410, which is transmitted by the client device 10 as connection request 411. When the client device 10 is connected to a customization device 14, the customization device 14 may generate a connection request 410, which is forwarded by the client device 10 as connection request 411. The intraoral scanning device 2 returns a connection response 412 which is received by the client device 10. The client device 10 may forward the connection response 412 to the customization device 14. The connection response 412 comprises an intraoral scanning device identifier 112 and/or a session identifier 180. The client device 10 generates one or more keys including a certificate key based on the intraoral scanning device identifier 112 and/or session identifier 180 received in the connection response and the client device key 182 stored in the memory unit. The client device 10 obtains and transmits authentication message 421 to the intraoral scanning device 2 based on the certificate key and the client device certificate 106. The authentication message 421 comprises encrypted client device certificate 106A. The encrypted client device certificate 106A is generated by encrypting the client device certificate 106 with the certificate key. The authentication message 421 comprises an authentication key identifier 166 indicative of the client device key 182 and/or authentication type identifier 168 indicative of the client device type identifier 156. Upon successful authentication of the authentication message 421 and/or verification, the client device 10 may receive an authentication response 422 from the intraoral scanning device 2. The client device 10 may forward the authentication response 422 to customization device 14. The communication channel is now open and secure. The client device 10 or customization device 14 via the client device 10 may send intraoral scanning device data 430 to the intraoral scanning device 2. Intraoral scanning device data 430 may comprise one or more of firmware, customization data, and/or intraoral scanning device operating parameters. Intraoral scanning device operation parameters may comprise volume control parameters, mode and/or program control parameters. Firmware may refer to a computer program provided by the intraoral scanning device manufacturer, and to be installed on the intraoral scanning device 2 to control the intraoral scanning device 2. Firmware is for example to be installed to upgrade the operations and capabilities of the intraoral scanning device 2. The client device 10 may transmit an authentication message 424 comprising a session key 188 to the customization device 14. The session key may be used for secure data communication 430 with the intraoral scanning device 2

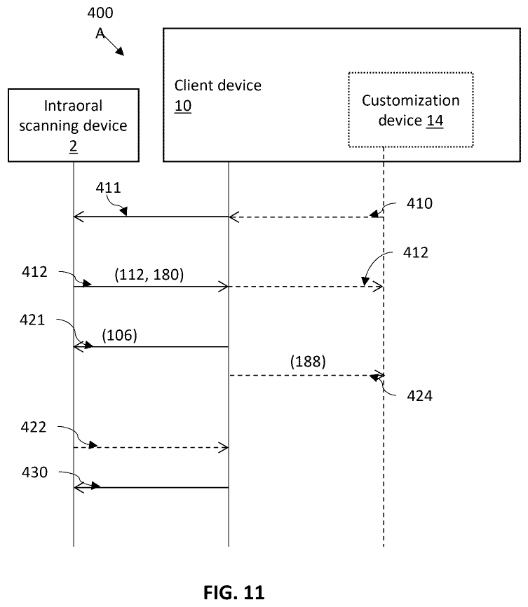

FIG. 11 schematically illustrates an exemplary signaling diagram 400A where the client device certificate 106 is included in the authentication message 421.

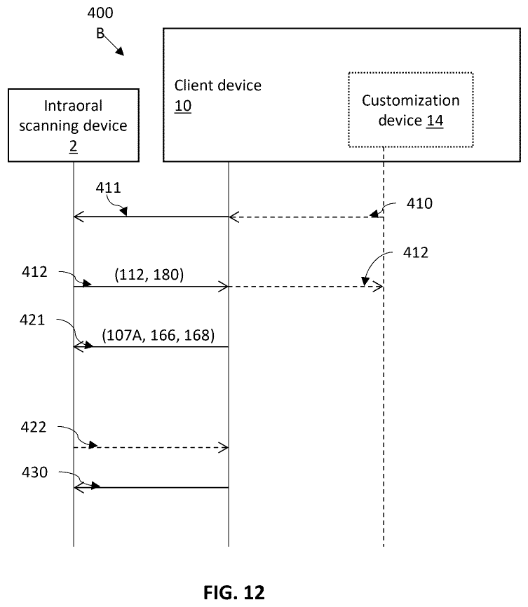

FIG. 12 schematically illustrates an exemplary signaling diagram 400B where the encrypted client device certificate 107A is included in the authentication message 421.

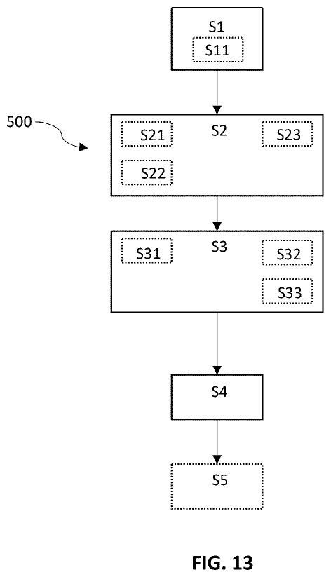

FIG. 13 schematically illustrates a flowchart of an exemplary method 500 of operating a client device for intraoral scanning device communication. The client device comprises a memory unit having a client device key and a client device certificate stored thereon. The method comprises receiving S1 a connection response comprising an intraoral scanning device identifier via the wireless interface; generating S2 one or more keys including a certificate key based on the intraoral scanning device identifier and the client device key; obtaining S3 an authentication message based on the certificate key and the client device certificate; and transmitting S4 the authentication message via the wireless interface. Obtaining S3 the authentication message comprises generating S31 an encrypted client device certificate by encrypting the client device certificate with the certificate key and including the encrypted client device certificate in the authentication message. The method 500 comprises obtaining S11 a session identifier as part of the connection response. Generating S2 one or more keys comprises generating S21 an intraoral scanning device key based on the intraoral scanning device identifier and the client device key, and generating S22 a common secret based on the intraoral scanning device key and the session identifier. The certificate key is based on the common secret and a certificate value. Generating S2 one or more keys optionally comprises generating S23 a session key based on the intraoral scanning device identifier, the session identifier and the client device key. The method may comprise transmitting S5 the session key to a customization device. In the method 500, the session key is based on the common secret and a session value. Obtaining S3 the authentication message comprises including S32 an authentication key identifier indicative of the client device key in the authentication message and/or including S33 an authentication type identifier in the authentication message.

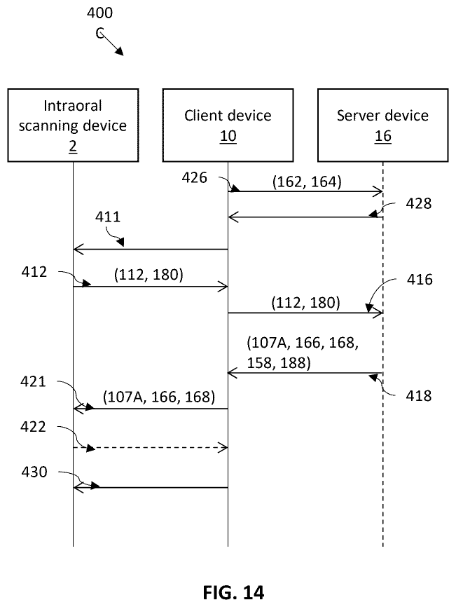

FIG. 14 schematically illustrates an exemplary signaling diagram 400C where obtaining the authentication message comprises obtaining an encrypted client device certificate from a server device. The client device 10 transmits a certificate request 416 to a server device 16. The certificate request 416 comprises the intraoral scanning device identifier 112 and the session identifier 180. The server device 16 obtains the client device certificate 107 from a memory unit thereof, calculates certificate key and session key based on the intraoral scanning device identifier 112 and the session identifier 180 and encrypts the client device certificate 107. The server device generates and transmits certificate response 418 to the client device 10. The certificate response 418 includes encrypted client device certificate 107A, authentication type identifier 168, authentication key identifier 166, client device identifier 158 and session key 188. The client device receives the certificate response 418 with the encrypted client device certificate 107A and includes the encrypted client device certificate in the authentication message. Thus, obtaining an encrypted client device certificate may comprise receiving a certificate response comprising the encrypted client device certificate from a server device. Optionally, the client device may, as illustrated in FIG. 14 be configured to perform a login procedure comprising transmitting a login request 426 comprising user identifier 162 and password 164. The server device 16 verifies the login request 426 and returns with login response 428 upon accept.

Although some embodiments have been described and shown in detail, the disclosure is not restricted to such details, but may also be embodied in other ways within the scope of the subject matter defined in the following claims. In particular, it is to be understood that other embodiments may be utilized, and structural and functional modifications may be made without departing from the scope of the present invention.

Benefits, other advantages, and solutions to problems have been described herein with regard to specific embodiments. However, the benefits, advantages, solutions to problems, and any component(s)/unit(s) that may cause any benefit, advantage, or solution to occur or become more pronounced are not to be construed as critical, required, or essential features or components/elements of any or all the claims or the invention. The scope of the invention is accordingly to be limited by nothing other than the appended claims, in which reference to an component/unit/element in the singular is not intended to mean “one and only one” unless explicitly so stated, but rather “one or more.” A claim may refer to any of the preceding claims, and “any” is understood to mean “any one or more” of the preceding claims.

It is Intended that the structural features of the devices described above, either in the detailed description and/or in the claims, may be combined with steps of the method, when appropriately substituted by a corresponding process.

As used, the singular forms “a,” “an,” and “the” are intended to include the plural forms as well (i.e. to have the meaning “at least one”), unless expressly stated otherwise. It will be further understood that the terms “includes,” “comprises,” “including,” and/or “comprising,” when used in this specification, specify the presence of stated features, integers, steps, operations, elements, and/or components, but do not preclude the presence or addition of one or more other features, integers, steps, operations, elements, components, and/or groups thereof. It will also be understood that when an element is referred to as being “connected” or “coupled” to another element, it can be directly connected or coupled to the other element but an intervening elements may also be present, unless expressly stated otherwise. Furthermore, “connected” or “coupled” as used herein may include wirelessly connected or coupled. As used herein, the term “and/or” includes any and all combinations of one or more of the associated listed items. The steps of any disclosed method is not limited to the exact order stated herein, unless expressly stated otherwise.

It should be appreciated that reference throughout this specification to “one embodiment” or “an embodiment” or “an aspect” or features included as “may” means that a particular feature, structure or characteristic described in connection with the embodiment is included in at least one embodiment of the disclosure. Furthermore, the particular features, structures or characteristics nay be combined as suitable in one or more embodiments of the disclosure, The previous description is provided to enable any person skilled in the art to practice the various aspects described herein. Various modifications to these aspects will be readily apparent to those skilled in the art, and the generic principles defined herein may be applied to other aspects.

The claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more.

### Item List:

1. A client device (10) configured to intraoral scanning device communication, the client device (10) comprising
a wireless interface (8) configured to receive 2D image data and/or 3D image data from an intraoral scanning device,
a memory unit (6);
a processing unit (4) configured to process the received 2D image data and/or 3D image data;
   the memory unit (6) having a client device key (182) and a client device certificate (106, 107) stored thereon,
   wherein the processing unit (4) is configured to:
receive a connection response (412) comprising an intraoral scanning device identifier (112) via the wireless interface (8);
generate one or more keys including a certificate key based on the intraoral scanning device identifier (112) and the client device key (182);
obtain an authentication message (424) based on the certificate key and the client device certificate (106, 107), and
transmit the authentication message (424) via the wireless interface (8).

2. Client device according to item 1, wherein the processing unit (4) is configured to obtain a session identifier (180), and wherein to generate one or more keys comprises to generate an intraoral scanning device key based on the intraoral scanning device identifier (112) and the client device key (182), and to generate a common secret based on the intraoral scanning device key and the session identifier (180).

3. Client device according to item 2, wherein the certificate key is based on the common secret and a certificate value.

4. Client device according to any of items 2-3, wherein to generate one or more keys comprises to generate a session key (188) based on the intraoral scanning device identifier (112), the session identifier (180) and the client device key (182), and wherein the processing unit (4) is configured to transmit the session key (188) to a customization device (14).

5. Client device according to item 4, wherein the session key (188) is based on the common secret and a session value.

6. Client device according to any of the preceding items, wherein the processing unit (4) is configured to include an authentication key identifier (166) indicative of the client device key (182) in the authentication message (424).

7. Client device according to any of the preceding items, wherein the processing unit (4) is configured to include an authentication type identifier (168) in the authentication message (424).

8. Client device according to any of the preceding items, wherein the client device certificate (106, 107) comprises one or more of:
a certificate type identifier (130);
a signing device identifier (136);
a client device type identifier (156);
a client device identifier (158);
a client device key identifier (159);
one or more hardware identifiers (148); and
a digital signature (113).

9. A method of operating a client device for intraoral scanning device communication, the client device comprising a memory unit (6) having a client device key (182) and a client device certificate (106, 107) stored thereon, the method comprising:
receiving (S1) a connection response (412) comprising an intraoral scanning device identifier (112) via the wireless interface;
generating (S2) one or more keys including a certificate key based on the intraoral scanning device identifier (112) and the client device key (182);
obtaining (S3) an authentication message (424) based on the certificate key and the client device certificate (106, 107), and
transmitting (S4) the authentication message (424) via the wireless interface (8).

10. Method according to item 9, the method comprising obtaining (S11) a session identifier (180), and wherein generating (S2) one or more keys comprises generating (S21) an intraoral scanning device key based on the intraoral scanning device identifier (112) and the client device key (182), and generating (S22) a common secret based on the intraoral scanning device key and the session identifier (180).

11. Method according to item 10, wherein the certificate key is based on the common secret and a certificate value.

12. Method according to any of items 10-11, wherein generating (S2) one or more keys comprises generating (S23) a session key (188) based on the intraoral scanning device identifier (112), the session identifier (180) and the client device key (182), and wherein the method comprises transmitting (S5) the session key (188) to a customization device (14).

13. Method according to item 12, wherein the session key (188) is based on the common secret and a session value.

14. Method according to any of items 9-13, wherein obtaining (S3) the authentication message (424) comprises including (S32) an authentication key identifier (166) indicative of the client device key (182) in the authentication message (424).

15. Method according to any of items 9-14, wherein obtaining (S3) the authentication message (424) comprises including (S33) an authentication type identifier (168) in the authentication message (424).

1. A handheld intraoral scanning device configured to acquire intraoral scan data from a three-dimensional dental object during a scanning session, the intraoral scanning device comprising;
a processing unit configured to process intraoral scan data of a patient and provide 3D image data;
a wireless interface configured to transmit the 3D image data; and
a memory unit,
wherein the processing unit is configured to:
receive a linking request for a session via the wireless interface, and wherein the session corresponds to accessing the memory unit of the handheld intraoral scanning device;
obtain a session identifier;
transmit, via the wireless interface, a linking response comprising an intraoral scanning device identifier and the session identifier;
receive, via the wireless interface, an authentication message comprising an authentication key identifier and client device data;
select an intraoral scanning device key from a plurality of intraoral scanning device keys in the memory unit based on the authentication key identifier;
verify the client device data based on the selected intraoral scanning device key; and
terminate the session if the verification fails.

2. A handheld intraoral scanning device according to claim 1, wherein the authentication message comprises an authentication type identifier, and wherein to select an intraoral scanning device key from a plurality of intraoral scanning device keys is based on the authentication type identifier.

3. A handheld intraoral scanning device according to claim 1, wherein the client device data comprises an encrypted client device certificate; and wherein the processing unit is configured to generate a certificate key based on a common secret; and wherein to verify the client device data comprises to decrypt the encrypted client device certificate with the certificate key to obtain a decrypted version of the encrypted client device certificate.

4. A handheld intraoral scanning device according to claim 3, wherein the common secret is based on the selected intraoral scanning device key and/or the session identifier.

5. A handheld intraoral scanning device according to claim 3, wherein to verify the client device data comprises to determine if the authentication key identifier matches a client device key identifier of the client device certificate, and wherein verification fails if no match is determined.

6. A handheld intraoral scanning device according to claim 3, wherein to verify the client device data comprises to determine if a client device type identifier of the client device certificate is valid and wherein verification fails if the client device type identifier of the client device certificate is not valid.

7. A handheld intraoral scanning device according to claim 3, wherein to verify the client device data comprises to verify a digital signature of the client device certificate, and wherein verification fails if the digital signature is not verified.

8. A handheld intraoral scanning device according to claim 3, wherein the client device certificate comprises a signing device identifier and/or a client device identifier, and wherein to verify the client device data comprises to determine if the signing device identifier and/or the client device identifier is valid and wherein verification fails if the client device identifier of the client device and/or the signing device identifier is not valid.

9. A handheld intraoral scanning device according to claim 3, wherein the processing unit is configured to receive an additional authentication message comprising client device data and an authentication device identifier, wherein the processing unit may be configured to
obtain, from the memory unit, the common secret based on the authentication device identifier;
generate an additional certificate key from the common secret; and
verify the client device data based on the additional certificate key.

10. A handheld intraoral scanning device according to claim 9, wherein the processing unit is configured to generate an offline session key based on the common secret and the session identifier, and wherein the processing unit is configured to communicate with the client device using the offline session key.

11. A handheld intraoral scanning device according to claim 3, wherein the authentication message comprises an authentication token identifier, and wherein the processing unit is configured to store the authentication token identifier in the memory unit and to link the authentication token identifier with the common secret.

12. A handheld intraoral scanning device according to claim 3, wherein the processing unit is configured to receive further authentication message comprising client device data, an authentication type identifier, an authentication key identifier and/or an authentication session token identifier, wherein the processing unit is configured to
obtain a common secret based on the authentication type identifier;
generate a token key based on the common secret;
generate a session token identifier based on the token key and the session identifier; and
verify the authentication session token identifier-based on the session token identifier.

13. A handheld intraoral scanning device according to claim 1, wherein the processing unit is configured to generate a session key based on the session identifier and the intraoral scanning device key, and wherein the processing unit is configured to receive and authenticate session data based on the session key.

14. A method of operating a handheld intraoral scanning device for acquiring intraoral scan data from a three-dimensional dental object during a scanning session, the intraoral scanning device comprising a processing unit configured to process intraoral scan data of a patient and provide 2D image data and/or 3D image data; a memory unit; and a wireless interface configured for transmitting the 2D image data and/or the 3D image data, wherein the method comprises the steps of:
receiving a linking request for a session via the wireless interface, and wherein the session corresponds to accessing the memory unit of the handheld intraoral scanning device;
obtaining a session identifier;
transmitting, via the wireless interface, a linking response comprising an intraoral scanning device identifier and the session identifier;
receiving, via the wireless interface, an authentication message comprising an authentication key identifier and client device data;
selecting an intraoral scanning device key from a plurality of intraoral scanning device keys based on the authentication key identifier;
verifying the client device data based on the selected intraoral scanning device key; and
terminating the session if verification fails.

15. A method according to claim 14, wherein the authentication message comprises an authentication type identifier, and wherein selecting an intraoral scanning device key from a plurality of intraoral scanning device keys is based on the authentication type identifier.
